SEQUENCE LISTING (3 sequences)

SEQ ID NO: 1 - DNA, 160 bp, Artificial Sequence
  source: /note=“Description of Artificial Sequence Synthetic polynucleotide”
aggagttcgcggttccaagtgatacactgatggcgttaggataagtttgaaacgcattaattgaaacaaggcaaattgctgtgtgagccgcctggaaaaggggagctacagggacctcgcaggaatacaacacagctcctaaaactggaattcgatttac

SEQ ID NO: 2 - DNA, 72 bp, Artificial Sequence
  source: /note=“Description of Artificial Sequence Synthetic oligonucleotide”
aggagttcgcggttccaagtgatacactgatggcgttaggataagtttgaaacgcattaattgaaacaaggc

SEQ ID NO: 3 - DNA, 59 bp, Artificial Sequence
  source: /note=“Description of Artificial Sequence Synthetic oligonucleotide”
cgcggttccaagtgatacactgatgggcmaagtttgaaacgcattaattgaaacaaggc

(19) United States
(12) Patent Application Publication
(10) Pub. No.: US 2023/0203573 A1
(43) Pub. Date: Jun. 29, 2023

(54) METHODS FOR DETECTION OF DONOR-DERIVED CELL-FREE DNA

(71) Applicant: Natera, Inc., San Carlos, CA (US)

(72) Inventors: Ryan SWENERTON, Millbrae, CA (US); Bernhard ZIMMERMANN, Manteca, CA (US); Ebad AHMED, San Jose, CA (US); Nathan LIANG, Daly City, CA (US); Allison RYAN, Belmont, CA (US); Fei LU, Fremont, CA (US); Paul VAN HUMMELEN, San Francisco, CA (US)

(73) Assignee: Natera, Inc., San Carlos, CA (US)

(21) Appl. No.: 17/925,693

(22) PCT Filed: May 27, 2021
(86) PCT No.: PCT/US21/34561

§ 371 Date: Nov. 16, 2022

Related U.S. Application Data
(60) Provisional application No. 63/031,879, filed on May 29, 2020.
(60) Provisional application No. 63/155,717, filed on Mar. 2, 2021.
(60) Provisional application No. 63/186,735, filed on May 10, 2021.

(51) Int. Cl. C12Q 1/6851; C12Q 1/686; C12Q 1/6876

(52) U.S. Cl. CPC C12Q 1/6851; C12Q 1/686; C12Q 1/6876; C12Q 2600/158

(57) ABSTRACT

The present disclosure provides methods for quantifying the amount of total cell-free DNA in a biological sample, comprising: isolating cell-free DNA from the biological sample, wherein a first Tracer DNA composition is added before or after isolation of the cell-free DNA; performing targeted amplification at 100 or more different target loci in a single reaction volume using 100 or more different primer pairs; sequencing the amplification products by high-throughput sequencing to generate sequencing reads; and quantifying the amount of total cell-free DNA using sequencing reads derived from the first Tracer DNA composition.

### BACKGROUND

Non-invasive monitoring using cell-free DNA (cfDNA) technology is an effective method for detecting nonself genotypes in prenatal (fetus), oncology (tumor), and transplantation (donor) applications. Furthermore, donor-derived cfDNA (dd-cfDNA) is a proven biomarker in transplantation (e.g., organ transplantation such as kidney and heart transplantation) for identifying active rejection. Existing commercial assays report dd-cfDNA results as a percentage of total cfDNA. However, results reported in this manner may not provide the most accurate depiction of rejection risk due to background cfDNA levels that can be affected by many factors. In some cases, atypically high levels of recipient cfDNA may lead to a decreased dd-cfDNA proportion, and a potential false negative interpretation. In addition, less frequently, lower than average cfDNA levels can lead to false positive results.

### SEQUENCE LISTING

The instant application contains a Sequence Listing which has been submitted electronically in ASCII format and is hereby incorporated by reference in its entirety. Said ASCII copy, created on May 26, 2021, is named N_033_WO_01_SL.txt and is 1,332 bytes in size.

### SUMMARY

In one aspect, the present invention relates to a method of quantifying the amount of total cell-free DNA in a biological sample, comprising: a) isolating cell-free DNA from the biological sample, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA composition.

In another aspect, the present invention relates to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

In a further aspect, the present invention relates to a method of determining the occurrence or likely occurrence of transplant rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of total cell-free DNA is quantified using sequencing reads derived from the first Tracer DNA composition, and determining the occurrence or likely occurrence of transplant rejection using the amount of donor-derived cell-free DNA by comparing the amount of donor-derived cell-free DNA to a threshold value, wherein the threshold value is determined according to the amount of total cell-free DNA.

In some embodiments, the threshold value is a function of the number of sequencing reads of the donor-derived cell-free DNA.

In some embodiments, the method further comprises flagging the sample if the amount of total cell-free DNA falls outside a pre-determined range. In some embodiments, the method further comprises flagging the sample if the amount of total cell-free DNA is above a pre-determined value. In some embodiments, the method further comprises flagging the sample if the amount of total cell-free DNA is below a pre-determined value.

In some embodiments, the method comprises adding the first Tracer DNA composition to a whole blood sample before plasma extraction. In some embodiments, the method comprises adding the first Tracer DNA composition to a plasma sample after plasma extraction and before isolation of the cell-free DNA. In some embodiments, the method comprises adding the first Tracer DNA composition to a composition comprising the isolated cell-free DNA. In some embodiments, the method comprises ligating adaptors to the isolated cell-free DNA to obtain a composition comprising adaptor-ligated DNA, and adding the first Tracer DNA composition to the composition comprising adaptor-ligated DNA.

In some embodiments, the method further comprises adding a second Tracer DNA composition before the targeted amplification. In some embodiments, the method further comprises adding a second Tracer DNA composition after the targeted amplification.

In some embodiments, the first and/or second Tracer DNA composition comprises a plurality of DNA molecules having different sequences.

In some embodiments, the first and/or second Tracer DNA composition comprises a plurality of DNA molecules having at different concentrations.

In some embodiments, the first and/or second Tracer DNA composition comprises a plurality of DNA molecules having different lengths. In some embodiments, the plurality of DNA molecules having different lengths are used to determine size distribution of the cell-free DNA in the sample.

In some embodiments, the first and/or second Tracer DNA composition comprises a plurality of DNA molecules of non-human origin.

In some embodiments, the first and/or second Tracer DNA composition each comprises a target sequence, wherein the target sequence comprises a barcode positioned between a pair of primer binding sites capable of binding to one of the primer pairs. In some embodiments, the barcode comprises reverse complement of a corresponding endogenous genome sequence capable of being amplified by the same primer pair.

In some embodiments, the ratio between the number of reads of the Tracer DNA and the number of reads of sample DNA is used to quantify the amount of total cell-free DNA. In some embodiments, the ratio between the number of reads of the barcode and the number of reads of the corresponding endogenous genome sequence is used to quantify the amount of total cell-free DNA.

In some embodiments, the target sequence is flanked on one or both sides by endogenous genome sequences. In some embodiments, the target sequence is flanked on one or both sides by non-endogenous sequences.

In some embodiments, the first and/or second Tracer DNA composition comprises synthetic double-stranded DNA molecules. In some embodiments, the first and/or second Tracer DNA composition comprises DNA molecules having a length of 50-500 bp. In some embodiments, the first and/or second Tracer DNA composition comprises DNA molecules having a length of 75-300 bp. In some embodiments, the first and/or second Tracer DNA composition comprises DNA molecules having a length of 100-250 bp. In some embodiments, the first and/or second Tracer DNA composition comprises DNA molecules having a length of 125-200 bp. In some embodiments, the first and/or second Tracer DNA composition comprises DNA molecules having a length of about 200 bp. In some embodiments, the first and/or second Tracer DNA composition comprises DNA molecules having a length of about 160 bp. In some embodiments, the first and/or second Tracer DNA composition comprises DNA molecules having a length of about 125 bp. In some embodiments, the first and/or second Tracer DNA composition comprises DNA molecules having a length of 500-1,000 bp.

In some embodiments, the targeted amplification comprises amplifying at least 100 polymorphic or SNP loci in a single reaction volume. In some embodiments, the targeted amplification comprises amplifying at least 200 polymorphic or SNP loci in a single reaction volume. In some embodiments, the targeted amplification comprises amplifying at least 500 polymorphic or SNP loci in a single reaction volume. In some embodiments, the targeted amplification comprises amplifying at least 1,000 polymorphic or SNP loci in a single reaction volume. In some embodiments, the targeted amplification comprises amplifying at least 2,000 polymorphic or SNP loci in a single reaction volume. In some embodiments, the targeted amplification comprises amplifying at least 5,000 polymorphic or SNP loci in a single reaction volume. In some embodiments, the targeted amplification comprises amplifying at least 10,000 polymorphic or SNP loci in a single reaction volume.

In some embodiments, each primer pair is designed to amplify a target sequence of about 35 to 200 bp. In some embodiments, each primer pair is designed to amplify a target sequence of about 50 to 100 bp. In some embodiments, each primer pair is designed to amplify a target sequence of about 60 to 75 bp. In some embodiments, each primer pair is designed to amplify a target sequence of about 65 bp.

In some embodiments, the transplant recipient is a human subject. In some embodiments, the transplant is a human transplant. In some embodiments, the transplant is a pig transplant. In some embodiments, the transplant is from a non-human animal.

In some embodiments, the transplant is an organ transplant, tissue transplant, or cell transplant. In some embodiments, the transplant is a kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, or blood transfusion.

In some embodiments, the method further comprises determine the transplant rejection as antibody mediated transplant rejection, T-cell mediated transplant rejection, graft injury, viral infection, bacterial infection, or borderline rejection. In some embodiments, the method further comprises determining the likelihood of one or more cancers. Cancer screening, detection, and monitoring are disclosed in PCT Patent Publication Nos. WO2015/164432, WO2017/181202, WO2018/083467, and WO2019/200228, each of which is incorporated herein by reference in its entirety. In other embodiments, the invention relates to screening a patient to determine their predicted responsiveness, or resistance, to one or more cancer treatments. This determination can be made by determining the existence of wild-type vs. mutated forms of a target gene, or in some cases the increased or over-expression of a target gene. Examples of such target screens include KRAS, NRAS, EGFR, ALK, KIT, and others. For example, a variety of KRAS mutations are appropriate for screening in accordance with the invention including, but not limited to, G12C, G12D, G12V, G13C, G13D, A18D, Q61H, K117N. In addition, PCT Patent Publication Nos. WO2015/164432, WO2017/181202, WO2018/083467, and WO2019/200228, which are incorporated herein by reference in their entirety.

In some embodiments, the method is performed without prior knowledge of donor genotypes. In some embodiments, the method is performed without prior knowledge of recipient genotypes. In some embodiments, the method is performed without prior knowledge of donor and/or recipient genotypes. In some embodiments, no genotyping of either the donor or the recipient is required prior to performing the method.

In some embodiments, the biological sample is a blood sample. In some embodiments, the biological sample is a plasma sample. In some embodiments, the biological sample is a serum sample. In some embodiments, the biological sample is a urine sample. In some embodiments, the biological sample is a sample of lymphatic fluid. In some embodiments, the sample is a solid tissue sample.

In some embodiments, the method further comprises longitudinally collecting a plurality of biological samples from the transplant recipient, and repeating steps (a) to (d) for each sample collected.

In some embodiments, the quantifying step comprises determining the percentage of donor-derived cell-free DNA out of the total of donor-derived cell-free DNA and recipient-derived cell-free DNA in the blood sample. In some embodiments, the quantifying step comprises determining the number of copies of donor-derived cell-free DNA. In some embodiments, the quantifying step comprises determining the number of copies of donor-derived cell-free DNA per volume unit of the blood sample.

In another aspect, the present invention relates to a method of diagnosing a transplant within a transplant recipient as undergoing acute rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of donor-derived cell-free DNA above a threshold value indicates that the transplant is undergoing acute rejection, wherein the threshold value is determined according to the amount of total cell-free DNA, and wherein the amount of total cell-free DNA is quantified using sequencing reads derived from the first Tracer DNA composition.

In another aspect, the present invention relates to a method of monitoring immunosuppressive therapy in a transplant recipient, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein a change in levels of donor-derived cell-free DNA over a time interval is indicative of transplant status, wherein the levels of donor-derived cell-free DNA is scaled according to the amount of total cell-free DNA, and wherein the amount of total cell-free DNA is quantified using sequencing reads derived from the first Tracer DNA composition.

In some embodiments, the method further comprises adjusting immunosuppressive therapy based on the levels of dd-cfDNA over the time interval.

In some embodiments, an increase in the levels of dd-cfDNA is indicative of transplant rejection and a need for adjusting immunosuppressive therapy. In some embodiments, no change or a decrease in the levels of dd-cfDNA indicates transplant tolerance or stability, and a need for adjusting immunosuppressive therapy.

In some embodiments, the method further comprises size selection to enrich for donor-derived cell-free DNA and reduce the amount of recipient-derived cell-free DNA disposed from bursting white-blood cells.

In some embodiments, the method further comprises a universal amplification step that preferentially amplifies donor-derived cell-free DNA over recipient-derived cell-free DNA originating from bursting or apoptosing white-blood cells.

In some embodiments, the method comprises longitudinally collecting a plurality of blood, plasma, serum, solid tissue, or urine samples from the transplant recipient after transplantation, and repeating steps (a) to (d) for each sample collected. In some embodiments, the method comprises collecting and analyzing blood, plasma, serum, solid tissue, or urine samples from the transplant recipient for a time period of about three months, or about six months, or about twelve months, or about eighteen months, or about twenty-four months, etc. In some embodiments, the method comprises collecting blood, plasma, serum, solid tissue, or urine samples from the transplant recipient at an interval of about one week, or about two weeks, or about three weeks, or about one month, or about two months, or about three months, etc.

In some embodiments, the determination that the amount of dd-cfDNA above a cutoff threshold is indicative of acute rejection of the transplant. Machine learning may be used to resolve rejection vs non-rejection. Machine learning is disclosed in WO2020/018522, titled “Methods and Systems for calling Ploidy States using a Neural Network” and filed on Jul. 16, 2019 as PCT/US2019/041981, which is incorporated herein by reference in its entirety. In some embodiments, the cutoff threshold value is scaled according to the amount of total cfDNA in the blood sample.

In some embodiments, the cutoff threshold value is expressed as percentage of dd-cfDNA (dd-cfDNA %) in the blood sample. In some embodiments, the cutoff threshold value is expressed as quantity or absolute quantity of dd-cfDNA. In some embodiments, the cutoff threshold value is expressed as quantity or absolute quantity of dd-cfDNA per volume unit of the blood sample. In some embodiments, the cutoff threshold value is expressed as quantity or absolute quantity of dd-cfDNA per volume unit of the blood sample multiplied by body mass, BMI, or blood volume of the transplant recipient.

In some embodiments, the cutoff threshold value takes into account the body mass, BMI, or blood volume of the patient. In some embodiments, the cutoff threshold value takes into account one or more of the following: donor genome copies per volume of plasma, cell-free DNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some embodiments, the method has a sensitivity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled or adjusted according to the amount of total cfDNA in the blood sample and a confidence interval of 95%.

In some embodiments, the method has a specificity of at least 50% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 60% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 70% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 80% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled or adjusted according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled or adjusted according to the amount of total cfDNA in the blood sample and a confidence interval of 95%.

In some embodiments, the transplant recipient has an elevated amount of total cell-free DNA. In some embodiments, the elevated amount of total cell-free DNA is caused by active viral infection. In some embodiments, the viral infection is COVID-19.

In some embodiments, the amount of donor-derived cell-free DNA is compared to a first and a second cutoff thresholds to determine the occurrence or likely occurrence of transplant rejection. In some embodiments, the first cutoff threshold is an estimated percentage of donor-derived cell-free DNA out of total cell-free DNA. In some embodiments, the first cutoff threshold is 0.8% dd-cfDNA, 0.9% dd-cfDNA, 1.0% dd-cfDNA, 1.1% dd-cfDNA, 1.2% dd-cfDNA, 1.3% dd-cfDNA, 1.4% dd-cfDNA, 1.5% dd-cfDNA, 1.6% dd-cfDNA, 1.7% dd-cfDNA, 1.8% dd-cfDNA, 1.9% dd-cfDNA, or 2.0% dd-cfDNA.

In some embodiments, the second cutoff threshold is absolute donor-derived cell-free DNA concentration. In some embodiments, the second cutoff threshold is 50 copies/ml, 55 copies/ml, 60 copies/ml, 65 copies/ml, 70 copies/ml, 71 copies/ml, 72 copies/ml, 73 copies/ml, 74 copies/ml, 75 copies/ml, 76 copies/ml, 77 copies/ml, 78 copies/ml, 79 copies/ml, 80 copies/ml, 81 copies/ml, 82 copies/ml, 83 copies/ml, 84 copies/ml, 85 copies/ml, 90 copies/ml, 95 copies/ml, or 100 copies/ml.

In some embodiments, the second cutoff threshold is calculated by multiplying the first cutoff threshold with a quant, wherein the quant is calculated by dividing the number of reads of total cell-free DNA by the number of reads of Tracer DNA per plasma volume. In some embodiments, the second cutoff threshold is 6.0 ml, 6.1 ml, 6.2 ml, 6.3 ml, 6.4 ml, 6.5 ml, 6.6 ml, 6.7 ml, 6.8 ml, 6.9 ml, 7.0 ml, 7.1 ml, 7.2 ml, 7.3 ml, 7.4 ml, 7.5 ml, 7.6 ml, 7.7 ml, 7.8 ml, 7.9 ml, 8.0 ml, 8.5 ml, 9.0 ml, 9.5 ml, or 10.0 ml.

In some embodiments, the method comprises calling rejection if the dd-cfDNA assay result exceeds the first cutoff threshold or the second cutoff threshold. In some embodiments, the method comprises calling non-rejection if the dd-cfDNA assay result is below the first cutoff threshold and the second cutoff threshold. In some embodiments, the method comprises calling rejection if (A) estimated dd-cfDNA %>0.8%, 0.9%, 1.0%, 1.1%, 1.2%, 1.3%, 1.4%, 1.5%, 1.6%, 1.7%, 1.8%, 1.9%, or 2.0%, or (B) dd-cfDNA concentration>50 copies/ml, 55 copies/ml, 60 copies/ml, 65 copies/ml, 70 copies/ml, 71 copies/ml, 72 copies/ml, 73 copies/ml, 74 copies/ml, 75 copies/ml, 76 copies/ml, 77 copies/ml, 78 copies/ml, 79 copies/ml, 80 copies/ml, 81 copies/ml, 82 copies/ml, 83 copies/ml, 84 copies/ml, 85 copies/ml, 90 copies/ml, 95 copies/ml, or 100 copies/ml. In some embodiments, the method comprises calling non-rejection if (A) estimated dd-cfDNA %<0.8%, 0.9%, 1.0%, 1.1%, 1.2%, 1.3%, 1.4%, 1.5%, 1.6%, 1.7%, 1.8%, 1.9%, or 2.0%, and (B) dd-cfDNA concentration<50 copies/ml, 55 copies/ml, 60 copies/ml, 65 copies/ml, 70 copies/ml, 71 copies/ml, 72 copies/ml, 73 copies/ml, 74 copies/ml, 75 copies/ml, 76 copies/ml, 77 copies/ml, 78 copies/ml, 79 copies/ml, 80 copies/ml, 81 copies/ml, 82 copies/ml, 83 copies/ml, 84 copies/ml, 85 copies/ml, 90 copies/ml, 95 copies/ml, or 100 copies/ml.

In some embodiments, the method comprises calling rejection if the dd-cfDNA assay result exceeds the first cutoff threshold or the second cutoff threshold. In some embodiments, the method comprises calling non-rejection if the dd-cfDNA assay result is below the first cutoff threshold and the second cutoff threshold. In some embodiments, the method comprises calling rejection if (A) estimated dd-cfDNA %>0.8%, 0.9%, 1.0%, 1.1%, 1.2%, 1.3%, 1.4%, 1.5%, 1.6%, 1.7%, 1.8%, 1.9%, or 2.0% or (B) estimated dd-cfDNA %×(total sample sequence reads/Tracer sequence reads/plasma volume)>6.0 ml, 6.1 ml, 6.2 ml, 6.3 ml, 6.4 ml, 6.5 ml, 6.6 ml, 6.7 ml, 6.8 ml, 6.9 ml, 7.0 ml, 7.1 ml, 7.2 ml, 7.3 ml, 7.4 ml, 7.5 ml, 7.6 ml, 7.7 ml, 7.8 ml, 7.9 ml, 8.0 ml, 8.5 ml, 9.0 ml, 9.5 ml, or 10.0 ml. In some embodiments, the method comprises calling non-rejection if (A) estimated dd-cfDNA %<0.8%, 0.9%, 1.0%, 1.1%, 1.2%, 1.3%, 1.4%, 1.5%, 1.6%, 1.7%, 1.8%, 1.9%, or 2.0% and (B) estimated dd-cfDNA %×(total sample sequence reads/Tracer sequence reads/plasma volume)<6.0 ml, 6.1 ml, 6.2 ml, 6.3 ml, 6.4 ml, 6.5 ml, 6.6 ml, 6.7 ml, 6.8 ml, 6.9 ml, 7.0 ml, 7.1 ml, 7.2 ml, 7.3 ml, 7.4 ml, 7.5 ml, 7.6 ml, 7.7 ml, 7.8 ml, 7.9 ml, 8.0 ml, 8.5 ml, 9.0 ml, 9.5 ml, or 10.0 ml.

In some embodiments, the first and second cutoff thresholds are combine into a single number or score. In some embodiments, the first and second cutoff thresholds are combined to produce one number or score and one cutoff such that this number or score is higher than its cutoff when either one of the two quantities (e.g., estimated dd-cfDNA % or dd-cfDNA concentration) (e.g., estimated dd-cfDNA % or estimated dd-cfDNA %×total cfDNA) is higher than its threshold, and the number or score is lower that its cutoff when both quantities are below their thresholds.

In some embodiments, the dd-cfDNA assay result is compared to a cutoff threshold to determine the occurrence or likely occurrence of transplant rejection, wherein the cutoff threshold is a function of the amount of donor-derived cell-free DNA and the amount of total cell-free DNA. In some embodiments, the dd-cfDNA assay result is compared to a cutoff threshold to determine the occurrence or likely occurrence of transplant rejection, wherein the cutoff threshold is a function of the number of reads of donor-derived cell-free DNA and the number of reads of total cell-free DNA.

In some embodiments, the function is a polynomial function. In some embodiments, the function is a logarithm function. In some embodiments, the function is an exponential function. In some embodiments, the function is a linear function. In some embodiments, the function is a nonlinear function.

In some embodiments, a transplant recipient is determined to have a high risk of transplant rejection if (ax\hat{ }n+by\hat{ }n)\hat{ }(1/n)>T, wherein: x=estimated dd-cfDNA %; y=estimated dd-cfDNA %×(number of reads of total cell-free DNA/number of reads of Tracer/plasma volume); a and b are each an arbitrary number; n is integer; T is a threshold value.

In some embodiments, a transplant recipient is determined to have a high risk of transplant rejection if log (ax\hat{ }n+by\hat{ }n)>T, wherein: x=estimated dd-cfDNA %; y=estimated dd-cfDNA %×(number of reads of total cell-free DNA/number of reads of Tracer/plasma volume); a and b are each an arbitrary number; n is integer; T is a threshold value.

In some embodiments, a transplant recipient is determined to have a high risk of transplant rejection if x×y>T, wherein: x=estimated dd-cfDNA %; y=estimated dd-cfDNA %×(number of reads of total cell-free DNA/number of reads of Tracer/plasma volume); T is a threshold value.

In some embodiments, a transplant recipient is determined to have a high risk of transplant rejection if ax−by>T, wherein: xx=estimated dd-cfDNA %; y=estimated dd-cfDNA %×(number of reads of total cell-free DNA/number of reads of Tracer/plasma volume); a and b are each an arbitrary number; T is a threshold value.

In some embodiments, the method comprises using an estimate percentage of donor-derived cell-free DNA in combination with a measurement of the total cell-free DNA concentration to determine the likelihood of organ failure. In some embodiments, the method comprises using an absolute donor-derived cell-free DNA concentration or a function thereof in combination with a measurement of the total cell-free DNA concentration to determine the likelihood of organ failure.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows an example workflow that uses Tracers to estimate the amount of total cfDNA, such as by comparing the number of sequence reads of the Tracers to the number of sequence reads of sample DNA or the number of sequence reads of a corresponding endogenous target, wherein the amount of total cfDNA can be used to adjust the threshold for calling transplant rejection status. In one example, a single Tracer at a single concentration is added to the sample. In another examples, multiple Tracers are added to the sample, such as Tracers of different lengths, Tracers at different concentrations, and Tracers introduced at different and/or multiple steps in the process. These new options can improve accuracy and precision, help quantify over a wider input range, assess efficiency of different steps at different size ranges, and calculate fragment size-distribution of input material.

FIG. 2 shows an example workflow that uses Tracers to estimate the amount of total cfDNA.

FIG. 3 shows an example design of Tracers, which is a 160 bp long DNA fragment derived from SNPs rs303935 and rs74720506. This Tracer is comprised of 80 bp sequence from both SNPs. The SNP nucleotide is replaced by a 9-nucleotide barcode. Tracer rs303935 amplicon length is 65 bp, while Panorama rs303935 amplicon length is 59 bp.

FIG. 4 shows two example designs of Tracers. Design 1 is the same as shown in FIG. 3, while Design 2 includes a reverse complement sequence of a corresponding endogenous target instead of an arbitrary 9-nucleotide barcode between forward and reverse primer binding sites.

FIG. 5 shows variability of background cfDNA levels, including distribution of cfDNA measurements observed in (i) pregnant women, (ii) kidney transplant recipients and (iii) early-stage cancer patients.

FIG. 6 shows concentration of background cfDNA in plasma is associated with patient weight as observed in (i) pregnant women and (ii) early stage cancer patients during surveillance period after completion of standard of care.

FIG. 7 shows levels of background cfDNA are elevated in patients undergoing active treatment and in metastatic cases (i); surgery transiently impacts cfDNA levels (ii).

FIG. 8 shows elevated background cfDNA levels can complicate rejection assessment in kidney transplant patients. Three cases with viral infections and clinical or subclinical rejections had dd-cfDNA proportions below 1% due to elevated background cfDNA levels.

FIG. 9 shows comparison between Tracer Metric, LabChip and Kapa qPCR (the outlier point in left panel LabChip data is excluded from R²).

FIG. 10 shows log plots comparison between Tracer Metric, LabChip and Kapa qPCR.

FIG. 11 shows consistent Tracer Metric when Prospera samples are run at both LDOR and HDOR (R²=0.99 with four high values excluded).

FIG. 12 shows percentage of dd-cfDNA in relation to Tracer Metric.

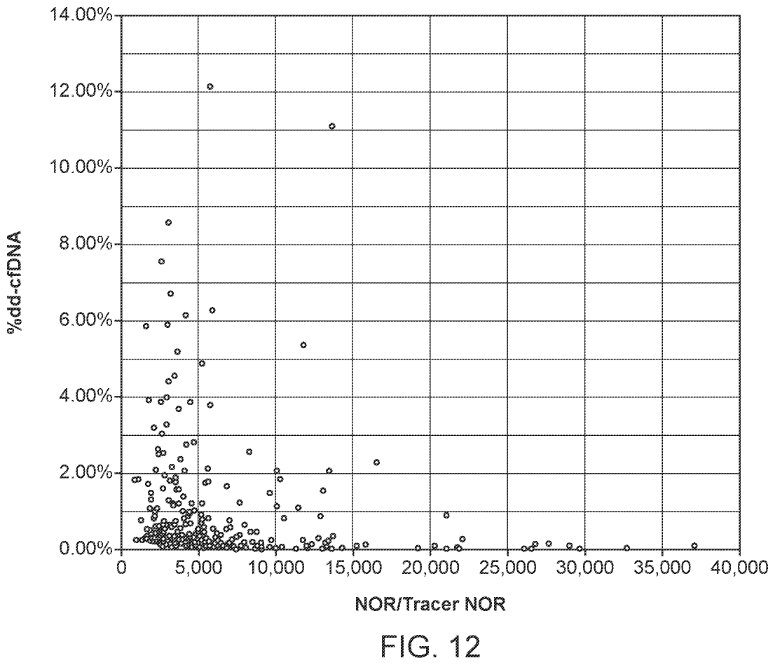

FIG. 13 shows histogram of Prospera Tracer Metric and Panorama Tracer Metric.

FIG. 14 shows histogram of Panorama cfDNA quantification and Panorama Tracer Metric.

FIG. 15 shows number of reads (NOR) of 95 individual Tracers.

FIG. 16 shows number of reads (NOR) of 10 individual Tracers.

FIG. 17 shows effects of background cfDNA on transplant rejection assessment.

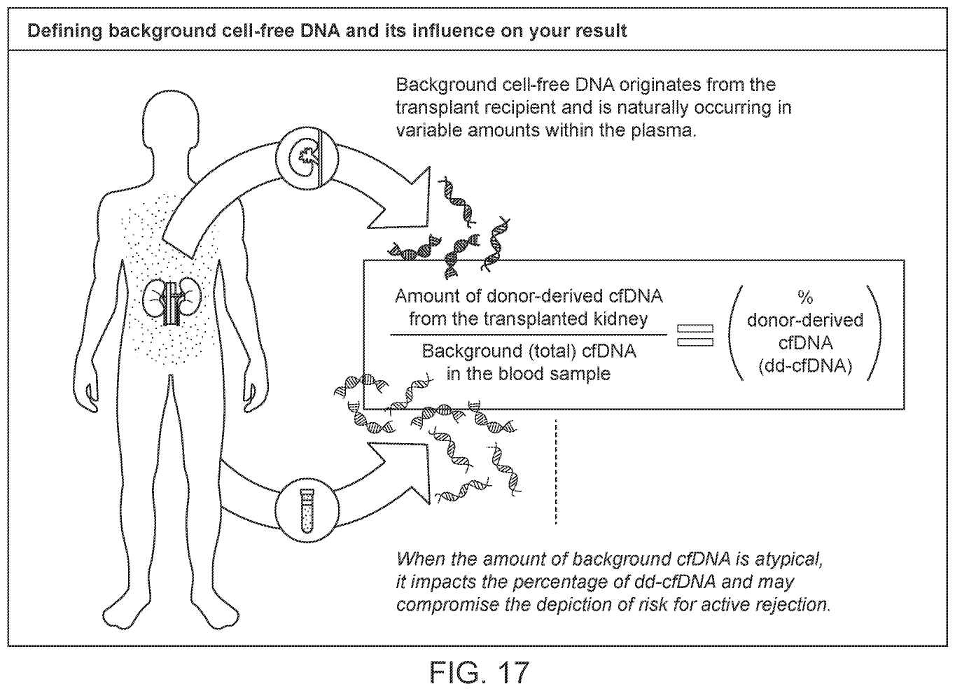

FIG. 18 shows donor-derived and total cfDNA levels in kidney transplant recipients with COVID-19. (A) Total cfDNA levels, represented as MoMs, were plotted against time in days from onset of COVID-19 symptoms to date of blood draw for dd-cfDNA tests at both the initial time point (yellow) and the follow-up time point (blue). (B) Total cfDNA levels at the initial time point (Draw 1) and the follow-up time pint (Draw 2), stratified by patients who had a single draw either due to death (red), or a second draw was unavailable (green), and patients with two draws (blue). Black lines connect paired tests. Grey dotted lines indicate medians for 15 paired values at first draw (6.2 MoM) and second draw (1.01 MoM). (C) dd-cfDNA levels at the initial time point and the follow-up time point, stratified as indicated in (B). Black lines connect paired tests. Grey dotted lines indicate medians for 15 paired values at first draw (0.2%) and second draw (0.32%).

FIG. 19 shows linear regression for COVID-19 severity. Relationship between total cfDNA (MoM) and WHO COVID19 Severity Score (Beta=0.06, SE=0.03, P=0.03).

FIG. 20 shows logistic regression for predicting mortality, in particular relationship between Total cfDNA (1st measurement) and probability of mortality (P=0.08, Beta=0.25, SE=0.14).

FIG. 21 shows logistic regression for predicting mortality, in particular relationship between dd-cfDNA and probability of mortality (P=0.08, Beta=−55.3, SE=31.3).

FIG. 22 shows an example embodiment of two-threshold methodology.

FIG. 23 shows improved detection of rejection in kidney transplant patients using an example two-threshold algorithm that combines donor fraction and absolute dd-cfDNA.

FIG. 24 shows an example embodiment of two-threshold methodology.

FIG. 25 shows improved detection of rejection in kidney transplant patients using an example two-threshold algorithm that combines donor fraction and absolute dd-cfDNA.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Sigdel et al., “Optimizing Detection of Kidney Transplant Injury by Assessment of Donor-Derived Cell-Free DNA via Massively Multiplex PCR,” J. Clin. Med. 8(1):19 (2019), is incorporated herein by reference in its entirety.

WO2020/010255, titled “METHODS FOR DETECTION OF DONOR-DERIVED CELL-FREE DNA” and filed on Jul. 3, 2019 as PCT/US2019/040603, is incorporated herein by reference in its entirety.

The methods described herein are, in some embodiments, powered by highly optimized, novel cfDNA technology and has now been enhanced with novel techniques that can quantify absolute background cfDNA in a streamlined manner. This improvement provides additional information for clinical decision making by identifying patients with atypical background cfDNA levels, and who might have a false negative result that could lead to a missed rejection.

The methods described herein assess all types of transplant rejection with great precision. From a single blood draw, certain embodiments of the methods described herein measure the amount of donor cfDNA from the transplanted organ in the patient's blood. Using a large number of single-nucleotide polymorphisms (SNP) (e.g., more than 13,000 SNPs) and advanced bioinformatics, these embodiments can differentiate donor and recipient cfDNA to provide a result as a percentage of dd-cfDNA in a transplant recipient's blood.

In some embodiments, the methods described herein incorporate (1) novel library preparation and/or (2) quantification of background cfDNA. In some embodiments, the library preparation technique results in higher yield, higher quality DNA than standard cfDNA tests. In some embodiments, it accounts for additional cfDNA that may be introduced to the sample during collection and transport. In some embodiments, the quantification of background cfDNA identifies atypical levels of background cfDNA that may influence the reported result for a particular patient. Applying both techniques can yield fewer false negative interpretations.

Disclosed herein are certain, non-exhaustive embodiments of methods for quantifying the amount of total cell-free DNA in a biological sample, as well methods for detection of transplant donor-derived cell-free DNA (dd-cfDNA) in a biological sample from a transplant recipient.

In one embodiment, the method relates to quantifying the amount of total cell-free DNA in a biological sample, comprising: a) isolating cell-free DNA from the biological sample, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA composition.

In another embodiment, the method relates to relates to quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

In another embodiment, the method relates to relates to determining the occurrence or likely occurrence of transplant rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of total cell-free DNA is quantified using sequencing reads derived from the first Tracer DNA composition, and determining the occurrence or likely occurrence of transplant rejection using the amount of donor-derived cell-free DNA by comparing the amount of donor-derived cell-free DNA to a threshold value, wherein the threshold value is determined according to the amount of total cell-free DNA.

### Definitions

Tracer DNA, or Internal Calibration DNA, refers to a composition of DNA for which one or more of the following is known advance—length, sequence, nucleotide composition, quantity, or biological origin. The tracer DNA can be added to a biological sample derived from a human subject to help estimate the amount of total cfDNA in said sample. It can also be added to reaction mixtures other than the biological sample itself.
Single Nucleotide Polymorphism (SNP) refers to a single nucleotide that may differ between the genomes of two members of the same species. The usage of the term does not imply any limit on the frequency with which each variant occurs.
Sequence refers to a DNA sequence or a genetic sequence. It may refer to the primary, physical structure of the DNA molecule or strand in an individual. It may refer to the sequence of nucleotides found in that DNA molecule, or the complementary strand to the DNA molecule. It may refer to the information contained in the DNA molecule as its representation in silico.
Locus refers to a particular region of interest on the DNA of an individual and includes without limitation one or more SNPs, the site of a possible insertion or deletion, or the site of some other relevant genetic variation. Disease-linked SNPs may also refer to disease-linked loci.
Polymorphic Allele, also “Polymorphic Locus,” refers to an allele or locus where the genotype varies between individuals within a given species. Some examples of polymorphic alleles include single nucleotide polymorphisms (SNPs), short tandem repeats, deletions, duplications, and inversions.
Allele refers to the nucleotides or nucleotide sequence occupying a particular locus.
Genetic Data also “Genotypic Data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.
Genetic Material also “Genetic Sample” refers to physical matter, such as tissue or blood, from one or more individuals comprising nucleic acids (e.g., comprising DNA or RNA)
Noisy Genetic Data refers to genetic data with any of the following: allele dropouts, uncertain base pair measurements, incorrect base pair measurements, missing base pair measurements, uncertain measurements of insertions or deletions, uncertain measurements of chromosome segment copy numbers, spurious signals, missing measurements, other errors, or combinations thereof.
Allelic Data refers to a set of genotypic data concerning a set of one or more alleles. It may refer to the phased, haplotypic data. It may refer to SNP identities, and it may refer to the sequence data of the nucleic acid, including insertions, deletions, repeats and mutations.
Allelic State refers to the actual state of the genes in a set of one or more alleles. It may refer to the actual state of the genes described by the allelic data.
Allelic Ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.
Allele Count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.
Primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow PCR amplification. A primer may also contain a molecular barcode. A primer may contain a random region that differs for each individual molecule.
Hybrid Capture Probe refers to any nucleic acid sequence, possibly modified, that is generated by various methods such as PCR or direct synthesis and intended to be complementary to one strand of a specific target DNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.
Sequence Read refers to data representing a sequence of nucleotide bases that were measured using a clonal sequencing method. Clonal sequencing may produce sequence data representing single, or clones, or clusters of one original DNA molecule. A sequence read may also have associated quality score at each base position of the sequence indicating the probability that nucleotide has been called correctly.
Mapping a sequence read is the process of determining a sequence read's location of origin in the genome sequence of a particular organism. The location of origin of sequence reads is based on similarity of nucleotide sequence of the read and the genome sequence.
DNA of Donor Origin refers to DNA that was originally part of a cell whose genotype was essentially equivalent to that of the transplant donor.
DNA of Recipient Origin refers to DNA that was originally part of a cell whose genotype was essentially equivalent to that of the transplant recipient.
Transplant recipient plasma refers to the plasma portion of the blood from a female from a patient who has received an allograft, e.g., an organ transplant recipient.
Preferential Enrichment of DNA that corresponds to a locus, or preferential enrichment of DNA at a locus, refers to any technique that results in the percentage of molecules of DNA in a post-enrichment DNA mixture that correspond to the locus being higher than the percentage of molecules of DNA in the pre-enrichment DNA mixture that correspond to the locus. The technique may involve selective amplification of DNA molecules that correspond to a locus. The technique may involve removing DNA molecules that do not correspond to the locus. The technique may involve a combination of methods. The degree of enrichment is defined as the percentage of molecules of DNA in the post-enrichment mixture that correspond to the locus divided by the percentage of molecules of DNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.
Amplification refers to a technique that increases the number of copies of a molecule of DNA.
Selective Amplification may refer to a technique that increases the number of copies of a particular molecule of DNA, or molecules of DNA that correspond to a particular region of DNA. It may also refer to a technique that increases the number of copies of a particular targeted molecule of DNA, or targeted region of DNA more than it increases non-targeted molecules or regions of DNA. Selective amplification may be a method of preferential enrichment.
Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.
Universal Adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.
Targeting refers to a method used to selectively amplify or otherwise preferentially enrich those molecules of DNA that correspond to a set of loci in a mixture of DNA.

### Tracer DNA and Use Thereof

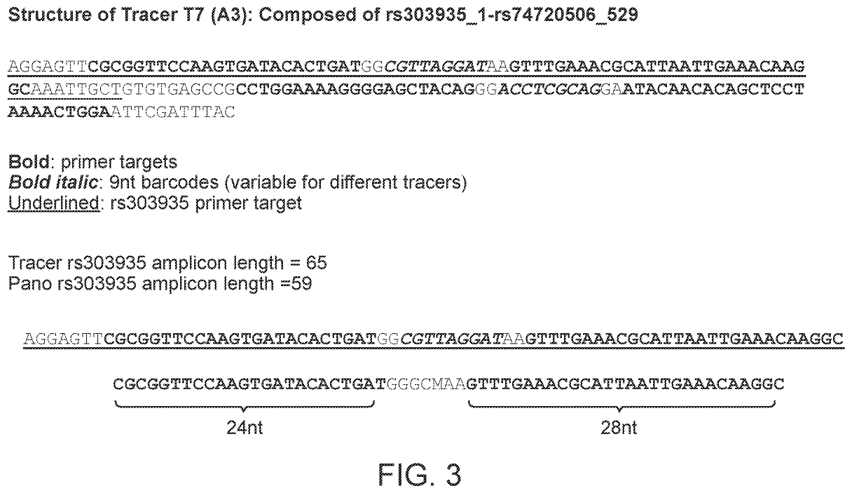

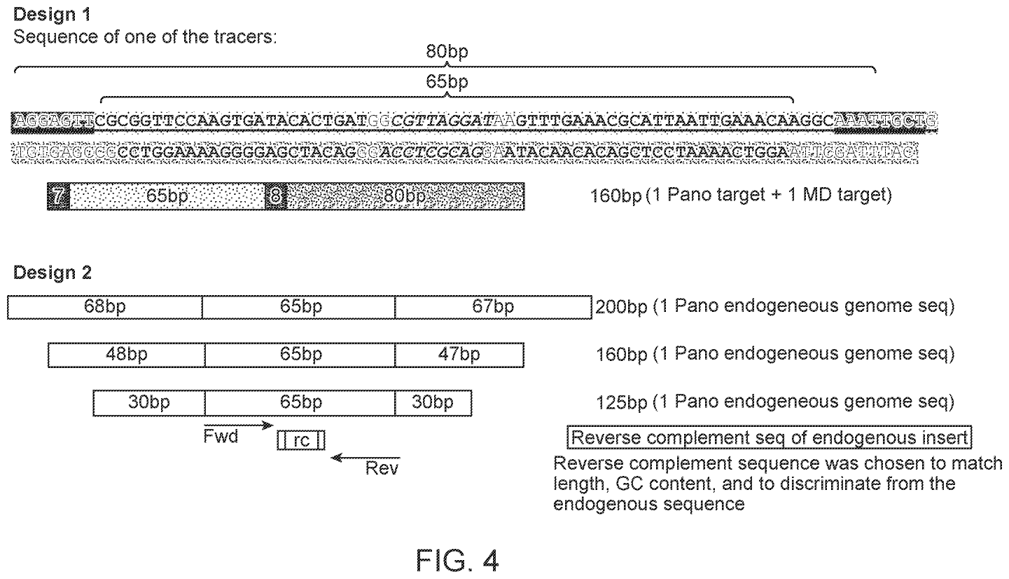

Examples of Tracer DNA are shown in FIG. 3 and FIG. 4. In some embodiments, the Tracer DNA comprises synthetic double-stranded DNA molecules. In some embodiments, the Tracer DNA comprises DNA molecules of non-human origin.

In some embodiments, the Tracer DNA comprises DNA molecules having a length of about 50-500 bp, or about 75-300 bp, or about 100-250 bp, or about 125-200 bp, or about 125 bp, or about 160 bp, or about 200 bp, or about 500-1,000 bp.

In some embodiments, the Tracer DNA comprises DNA molecules having the same or substantially the same length, such as a DNA molecule having a length of about 125 bp, or about 160 bp, or about 200 bp. In some embodiments, the Tracer DNA comprises DNA molecules having different lengths, such as a first DNA molecule having a length of about 125 bp, a second DNA molecule having a length of about 160 bp, and a third DNA molecule having a length of about 200 bp. In some embodiments, the DNA molecules having different lengths are used to determine size distribution of the cell-free DNA in the sample

In some embodiments, the Tracer DNA comprises a target sequence, wherein the target sequence comprises a barcode positioned between a pair of primer binding sites capable of binding to a pair of primers. In some embodiments, at least part of the Tracer DNA is designed based on an endogenous human SNP locus, by replacing an endogenous sequence containing the SNP locus with the barcode. During the mmPCR target enrichment step, the primer pair targeting the SNP locus can also amplify the portion of Tracer DNA containing the barcode.

In some embodiments, the barcode is an arbitrary barcode. In some embodiments, the barcode comprises reverse complement of a corresponding endogenous genome sequence capable of being amplified by the same primer pair.

In some embodiments, the target sequence within the Tracer DNA is flanked on one or both sides by endogenous genome sequences. In some embodiments, the target sequence within the Tracer DNA is flanked on one or both sides by non-endogenous sequences.

In some embodiments, the Tracer DNA comprises a plurality of target sequences. In some embodiments, the Tracer DNA comprises a first target sequence comprising a first barcode positioned between a first pair of primer binding sites capable of binding to a first pair of primers, and a second barcode positioned between a second pair of primer binding sites capable of binding to a second pair of primers. In some embodiments, the first and/or second target sequence is designed based on one or more endogenous human SNP loci, by replacing an endogenous sequence containing a SNP locus with a barcode. In some embodiments, the first and/or second barcode is an arbitrary barcode. In some embodiments, the first and/or second barcode comprises reverse complement of a corresponding endogenous genome sequence capable of being amplified by the first or second primer pair. In some embodiments, the first and/or second target sequence within the Tracer DNA is flanked on one or both sides by endogenous genome sequences. In some embodiments, the first and/or second target sequence within the Tracer DNA is flanked on one or both sides by non-endogenous sequences.

In some embodiments, the Tracer DNA comprises DNA molecules having the same or substantially the same sequence, such as the Tracer DNA sequence shown in FIG. 3. In some embodiments, the Tracer DNA comprises DNA molecules having different sequences.

In some embodiments, the Tracer DNA comprises a first DNA comprising a first target sequence and a second DNA comprising a second target sequence. In some embodiments, the first target sequence and second target sequence have different barcodes positioned between the same primer binding sites. In some embodiments, the first target sequence and second target sequence have different barcodes positioned between the same primer binding sites, wherein the different barcodes have the same or substantially the same lengths. In some embodiments, the first target sequence and second target sequence have different barcodes positioned between the same primer binding sites, wherein the different barcodes have different lengths. In some embodiments, the first target sequence and second target sequence are designed based on different endogenous human SNP loci, and hence comprise different primer binding sites. In some embodiments, the amount of first DNA and the amount of the second DNA are the same or substantially the same in the Tracer DNA. In some embodiments, the amount of first DNA and the amount of the second DNA are different in the Tracer DNA.

### Determining Amount of Total Cell-Free DNA Using Tracer DNA

In certain embodiments, the Tracer DNA can be used to improve accuracy and precision of the method described herein, help quantify over a wider input range, assess efficiency of different steps at different size ranges, and/or calculate fragment size-distribution of input material.

Some embodiments of the present invention relate to a method of quantifying the amount of total cell-free DNA in a biological sample, comprising: a) isolating cell-free DNA from the biological sample, wherein a first Tracer DNA is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA.

In some embodiments, the method comprises adding the first Tracer DNA to a whole blood sample before plasma extraction. In some embodiments, the method comprises adding the first Tracer DNA to a plasma sample after plasma extraction and before isolation of the cell-free DNA. In some embodiments, the method comprises adding the first Tracer DNA to a composition comprising the isolated cell-free DNA. In some embodiments, the method comprises ligating adaptors to the isolated cell-free DNA to obtain a composition comprising adaptor-ligated DNA, and adding the first Tracer DNA to the composition comprising adaptor-ligated DNA.

In some embodiments, the method further comprises adding a second Tracer DNA before the targeted amplification. In some embodiments, the method further comprises adding a second Tracer DNA after the targeted amplification.

In some embodiments, the amount of total cfDNA in the sample is estimated using the NOR of the Tracer DNA (identifiable by the barcode), the NOR of sample DNA, and the known amount of the Tracer DNA added to the plasma sample. In some embodiments, the ratio between the NOR of the Tracer DNA and the NOR of sample DNA is used to quantify the amount of total cell-free DNA. In some embodiments, the ratio between the NOR of the barcode and the NOR of the corresponding endogenous genome sequence is used to quantify the amount of total cell-free DNA. In some embodiments, this information along with the plasma volume can also be used to calculate the amount of cfDNA per volume of plasma. In some embodiments, these can be multiplied by the percentage of donor DNA to calculate the total donor cfDNA and the donor cfDNA per volume of plasma.

### Adjusting Threshold for Calling Transplant Rejection Using Amount of Total Cell-Free DNA

Some embodiments of the present invention relate to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

Some embodiments use either a fixed threshold of donor DNA per plasma volume or one that is not fixed, such as adjusted or scaled as noted herein. The way that this is determined can be based on using a training data set to build an algorithm to maximize performance. It may also take into account other data such as patient weight, age, or other clinical factors.

In some embodiments, the method further comprises determining the occurrence or likely occurrence of transplant rejection using the amount of donor-derived cell-free DNA. In some embodiments, the amount of donor-derived cell-free DNA is compared to a cutoff threshold value to determine the occurrence or likely occurrence of transplant rejection, wherein the cutoff threshold value is adjusted or scaled according to the amount of total cell-free DNA. In some embodiments, the cutoff threshold value is a function of the number of reads of the donor-derived cell-free DNA.

In some embodiments, the method comprises applying a scaled or dynamic threshold metric that takes into account the amount of total cfDNA in the samples to more accurately assess transplant rejection. In some embodiments, the method further comprises flagging the sample if the amount of total cell-free DNA is above a pre-determined value. In some embodiments, the method further comprises flagging the sample if the amount of total cell-free DNA is below a pre-determined value.

### Multiplex Amplification

In some embodiments, the method comprises performing a multiplex amplification reaction to amplify a plurality of polymorphic loci in one reaction mixture before determining the sequences of the selectively enriched DNA.

In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 100; 200; 500; 1,000; 2,000; 5,000; 10,000; 20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to at least 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify cfDNA obtained from a blood, plasma, serum, solid tissue, or urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some embodiments, the sequences of the amplicons are determined by performing high-throughput sequencing.

The genetic data of the transplanted organ and/or of the transplant recipient can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, or any other sequencing method. In some embodiments, the high throughput sequencing is performed on Illumina NextSeq®, followed by demultiplexing and mapping to the human reference genome. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

### WORKING EXAMPLES

### Example 1

The workflow of this non-limiting example corresponds to the workflow disclosed in Sigdel et al., “Optimizing Detection of Kidney Transplant Injury by Assessment of Donor-Derived Cell-Free DNA via Massively Multiplex PCR,” J. Clin. Med. 8(1):19 (2019), which is incorporated herein by reference in its entirety. This example is illustrative only, and a skilled artisan will appreciate that the invention disclosed herein can be practiced in a variety of other ways.

Blood Samples

Male and female adult or young-adult patients received a kidney from related or unrelated living donors, or unrelated deceased donors. Time points of patient blood draw following transplantation surgery were either at the time of an allograft biopsy or at various pre-specified time intervals based on lab protocols. Typically, samples were biopsy-matched and had blood drawn at the time of clinical dysfunction and biopsy or at the time of protocol biopsy (at which time most patients did not have clinical dysfunction). In addition, some patients had serial post transplantation blood drawn. The selection of study samples was based on (a) adequate plasma being available, and (b) if the sample was associated with biopsy information. Among the full 300 sample cohort, 72.3% were drawn on the day of biopsy.

Dd-cfDNA Measurement in Blood Samples

Cell-free DNA was extracted from plasma samples using the QIAamp Circulating Nucleic Acid Kit (Qiagen) and quantified on the LabChip NGS 5k kit (Perkin Elmer, Waltham, Mass., USA) following manufacturer's instructions. Cell-free DNA was input into library preparation using the Natera Library Prep kit as described in Abbosh et al, Nature 545: 446-451 (2017), with a modification of 18 cycles of library amplification to plateau the libraries. Purified libraries were quantified using LabChip NGS 5k as described in Abbosh et al, Nature 545: 446-451 (2017). Target enrichment was accomplished using massively multiplexed-PCR (mmPCR) using a modified version of a described in Zimmermann et al., Prenat. Diagn. 32:1233-1241 (2012), with 13,392 single nucleotide polymorphisms (SNPs) targeted. Amplicons were then sequenced on an Illumina HiSeq 2500 Rapid Run®, 50 cycles single end, with 10-11 million reads per sample.

Statistical Analyses of Dd-cfDNA and eGFR

In each sample, dd-cfDNA was measured and correlated with rejection status, and results were compared with eGFR. Where applicable, all statistical tests were two sided. Significance was set at p<0.05. Because the distribution of dd-cfDNA in patients was severely skewed among the groups, data were analyzed using a Kruskal-Wallis rank sum test followed by Dunn multiple comparison tests with Holm correction. eGFR (serum creatinine in mg/dL) was calculated as described previously for adult and pediatric patients. Briefly, eGFR=186×Serum Creatinine^{-1.154}×Age^{-0.203}×(1.210 if Black)×(0.742 if Female).

To evaluate the performance of dd-cfDNA and eGFR (mL/min/1.73 m²) as rejection markers, samples were separated into an AR group and a non-rejection group (BL+STA+OI). Using this categorization, the following predetermined cut-offs were used to classify a sample as AR: >1% for dd-cfDNA and <60.0 for eGFR.

To calculate the performance parameters of each marker (sensitivity, specificity, positive predictive value (PPV), negative predictive value (NPV), and area under the curve (AUC)), a bootstrap method was used to account for repeated measurements within a patient. Briefly, at each bootstrap step, a single sample was selected from each patient; by assuming independence among patients, the performance parameters and their standard errors were calculated. This was repeated 10,000 times; final confidence intervals were calculated using the bootstrap mean for the parameter with the average of the bootstrap standard errors with standard normal quantiles. Standard errors for sensitivity and specificity were calculated assuming a binomial distribution; for PPV and NPV a normal approximation was used; and for AUC the DeLong method was used. Performance was calculated for all samples with a matched biopsy, including the sub-cohort consisting of samples drawn at the same time as a protocol biopsy.

Differences in dd-cfDNA levels by donor type (living related, living non-related, and deceased non-related) were also evaluated. Significance was determined using the Kruskal-Wallis rank sum test as described above. Inter- and intra-variability in dd-cfDNA over time was evaluated using a mixed effects model with a logarithmic transformation on dd-cfDNA; 95% confidence intervals (CI) for the intra- and inter-patient standard deviations were calculated using a likelihood profile method.

Post hoc analyses evaluated (a) different dd-cfDNA thresholds to maximize NPV and (b) combined dd-cfDNA and eGFR to define an empirical rejection zone that may improve the PPV for AR diagnosis. All analyses were done using R 3.3.2 using the FSA (for Dunn tests), lme4 (for mixed effect modeling) and pROC (for AUC calculations) packages.

Biopsy Samples

Optionally, kidney biopsies were analyzed in a blinded manner by a pathologist and were graded by the 2017 Banff classification for active rejection (AR); intragraft C4d stains were performed to assess for acute humoral rejection. Biopsies were not done in cases of active urinary tract infection (UTI) or other infections. Transplant “injury” was defined as a >20% increase in serum creatinine from its previous steady-state baseline value and an associated biopsy that was classified as either active rejection (AR), borderline rejection (BL), or other injury (OI) (e.g., drug toxicity, viral infection). Active rejection was defined, at minimum, by the following criteria: (1) T-cell-mediated rejection (TCMR) consisting of either a tubulitis (t) score >2 accompanied by an interstitial inflammation (i) score >2 or vascular changes (v) score >0; (2) C4d positive antibody-mediated rejection (ABMR) consisting of positive donor specific antibodies (DSA) with a glomerulitis (g) score >0/or peritubular capillaritis score (ptc)>0 or v>0 with unexplained acute tubular necrosis/thrombotic micro angiopathy (ATN/TMA) with C4d=2; or (3) C4d negative ABMR consisting of positive DSA with unexplained ATN/TMA with g+ptc≥2 and C4d is either 0 or 1. Borderline change (BL) was defined by t1+i0, or t1+i1, or t2+i0 without explained cause (e.g., polyomavirus-associated nephropathy (PVAN)/infectious cause/ATN). Other criteria used for BL changes were g>0 and/or ptc>0, or v>0 without DSA, or C4d or positive DSA, or positive C4d without nonzero g or ptc scores. Normal (STA) allografts were defined by an absence of significant injury pathology as defined by Banff schema.

### Example 2

This example is illustrative only, and a skilled artisan will appreciate that the invention disclosed herein can be practiced in a variety of other ways.

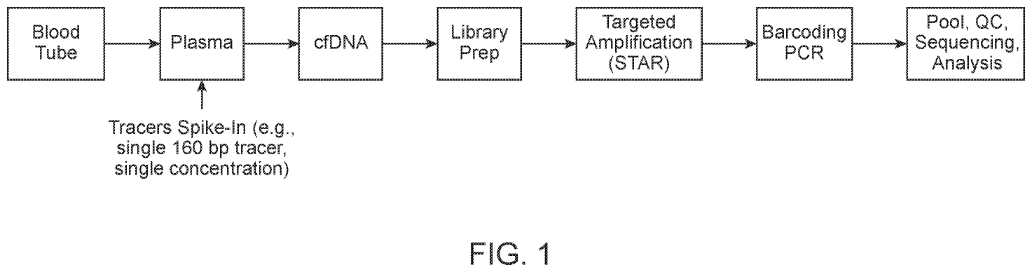

The workflow described in Example 1 is modified by adding a 160-bp Tracer DNA to the plasma sample prior to extraction of cell-free DNA, as shown in FIG. 1. The structure of this Tracer DNA is shown in Design 1 of FIG. 4, which is derived from SNPs rs303935 and rs74720506. The portion of the Tracer DNA based on SNP rs303935 is modified to replace a 3-nucleotide endogenous sequence containing the SNP locus (GCM) with a 9-nucleotide barcode (CGTTAGGAT). During the mmPCR target enrichment step, the primer pairs targeting SNP rs303935 also amplify the Tracer DNA. The amount of total cfDNA in the sample is estimated using the number of sequences reads of the Tracer DNA (identifiable by the barcode), the number of sequence reads of sample DNA, and the known amount of the Tracer DNA added to the plasma sample.

### Example 3

This example is illustrative only, and a skilled artisan will appreciate that the invention disclosed herein can be practiced in a variety of other ways.

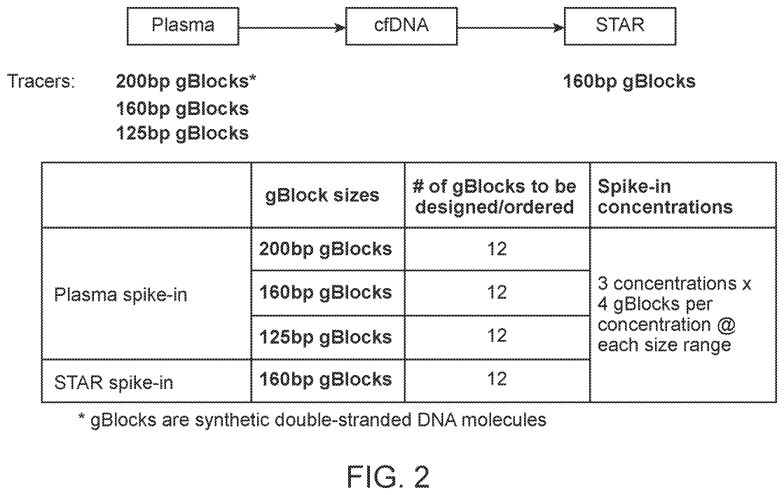

The workflow described in Example 1 is modified by adding a 200-bp Tracer DNA, a 160-bp Tracer DNA, and a 125-bp Tracer DNA to the plasma sample prior to extraction of cell-free DNA, as shown in FIG. 2. The structures of the 3 Tracer DNA are shown in Design 2 of FIG. 4, each of which is derived from a SNP locus. The portion of the Tracer DNA based on the SNP locus is modified to replace an endogenous sequence containing the SNP locus with a barcode corresponding to the reverse complement of the endogenous sequence. During the mmPCR target enrichment step, the primer pairs targeting the SNP locus also amplify the Tracer DNA. The amount of total cfDNA in the sample is estimated using the number of sequences reads of the Tracer DNA (identifiable by the barcode), the number of sequences reads of sample DNA, and the known amount of the Tracer DNA added to the plasma sample. As the 3 Tracer DNAs have different lengths, their NORs can also be used to estimate size distribution of the cfDNA in the plasma sample.

### Example 4

This example is illustrative only, and a skilled artisan will appreciate that the invention disclosed herein can be practiced in a variety of other ways.

Three methods were used to evaluate workflows that are capable of screening for high cfDNA outliers—Tracer Metric, Kapa qPCR, and LabChip. Tracer Metric and qPCR were compared with LabChip as the orthogonal method. All three methods were divided by the plasma volume to measure yield.

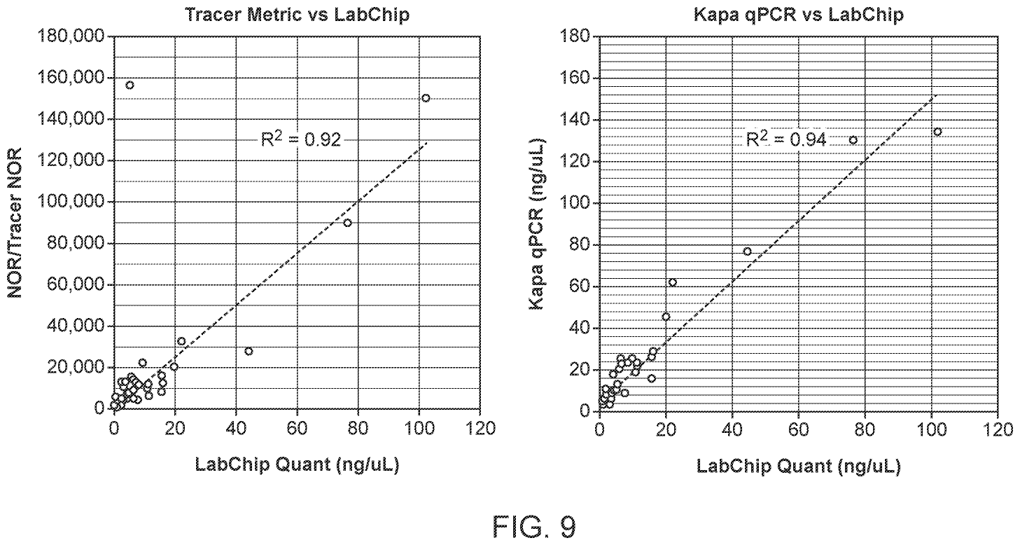

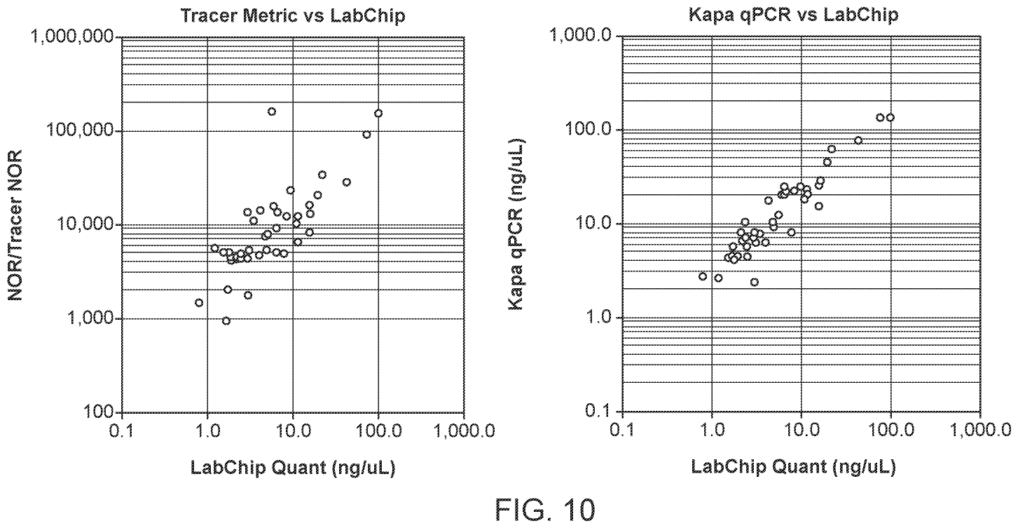

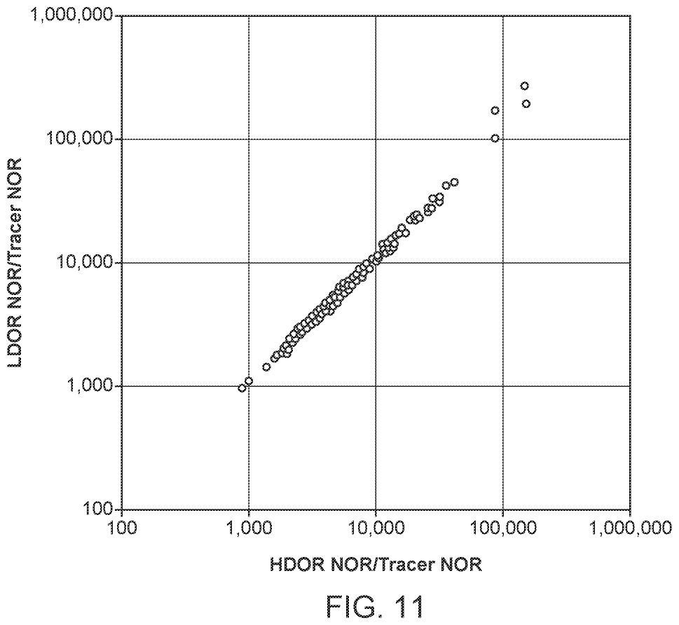

A total 45 commercial Prospera samples were quantified by Tracer Metric, qPCR (triplicate), and LabChip (triplicate). Quant methods were correlated at both high and low cfDNA concentrations. As shown in FIG. 9 and FIG. 10, both Tracer Metric and qPCR have R²>0.9 (Kapa intra-replicate R²=0.93; LabChip intra-replicate R²=0.94). FIG. 11 shows consistent Tracer Metric NOR when Prospera samples are run at both LDOR and HDOR (R²=0.99). Tracer Metric correlates very well between the two indicating it is stable in our processing.

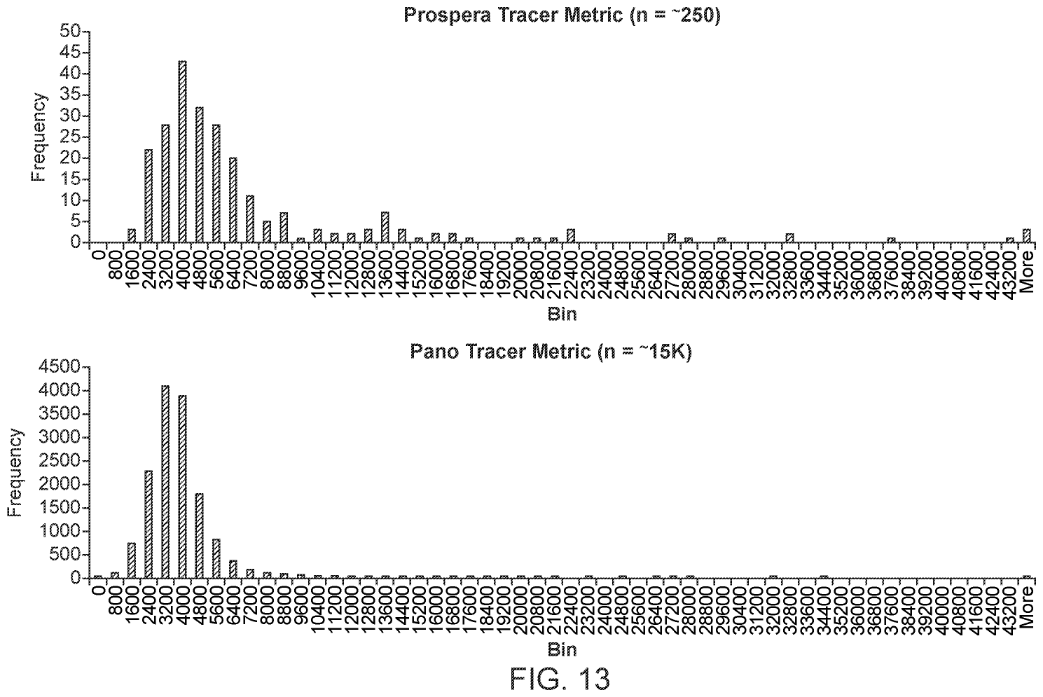

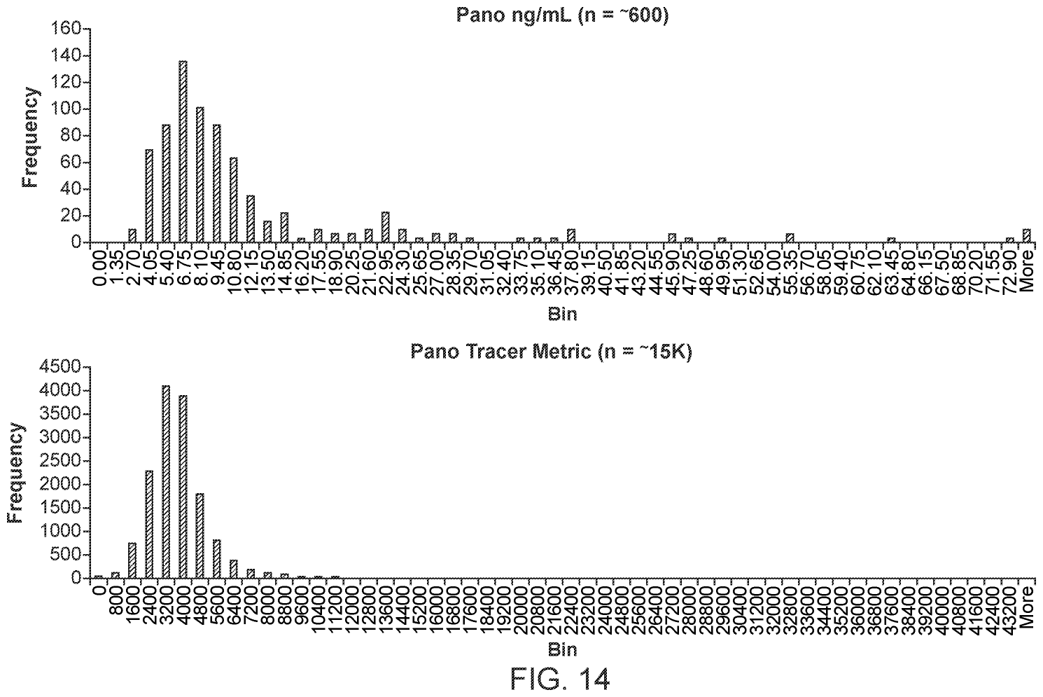

FIG. 13 shows histogram of Prospera Tracer Metric and Panorama Tracer Metric, based on retrospective analysis of commercial data. High outliers are present in Prospera that are not observed in Panorama. 3% of Prospera samples are >7× the median (vs 0.1% of Pano samples). FIG. 14 shows histogram of Panorama cfDNA quantification and Panorama Tracer Metric. Panorama Tracer Metric distribution mirrors the concentration distribution on both the high and low ends.

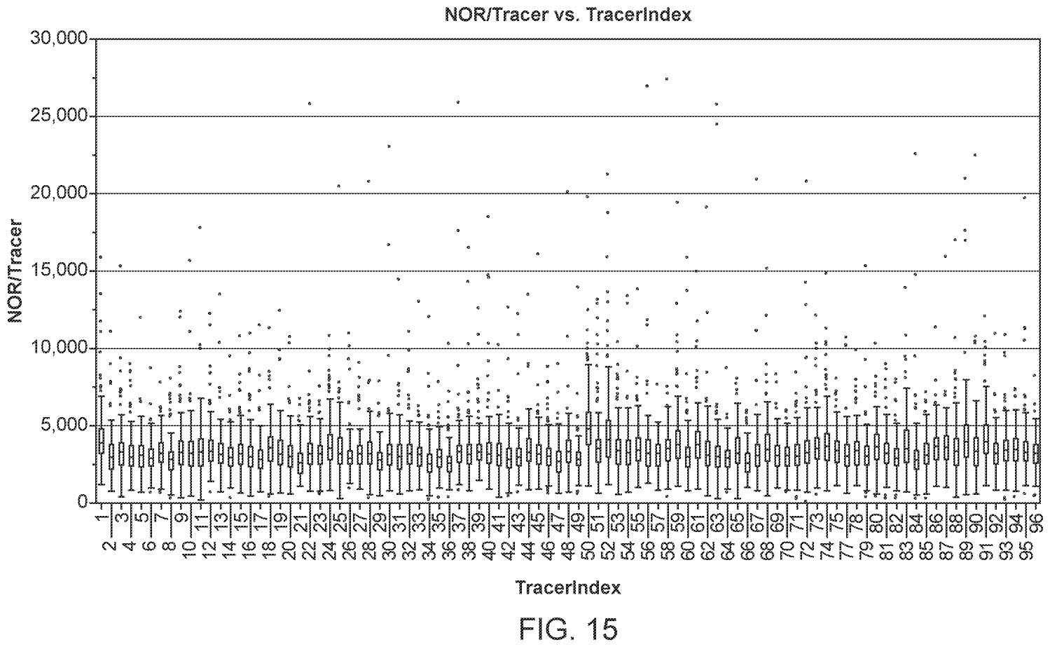

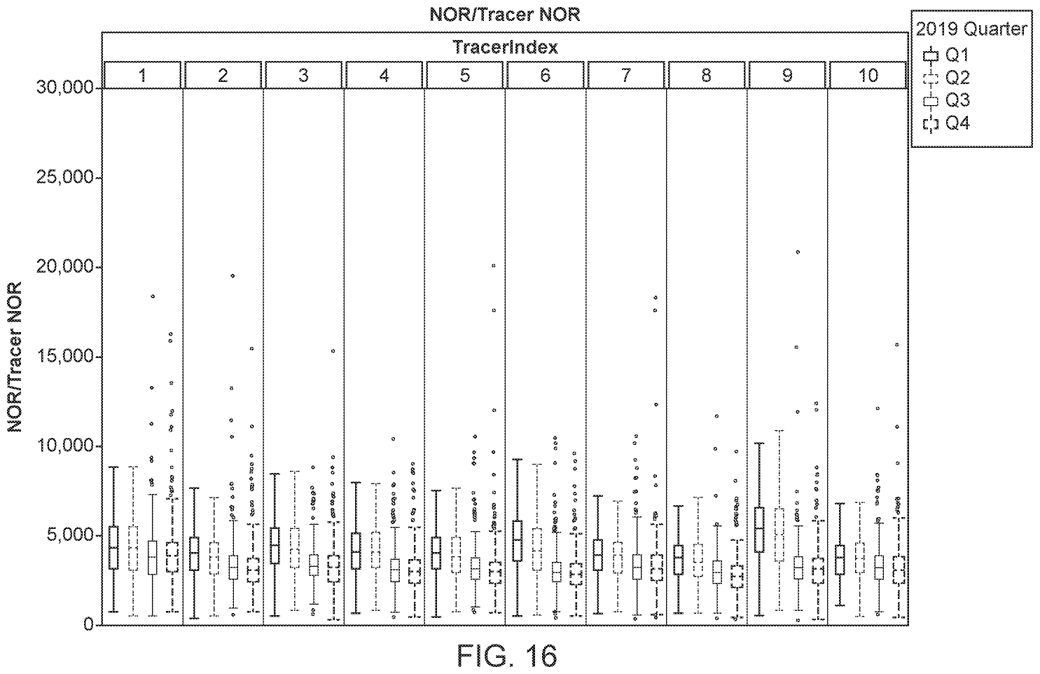

FIG. 15 shows number of reads (NOR) of 95 individual Tracers, based on retrospective analysis of commercial data. All 95 tracers perform similarly, with about −150 data points per tracer. Outliers are not clustered with individual tracers. FIG. 16 shows number of reads (NOR) of 10 individual Tracers split by the quarter, with about −300 data points per tracer. The performance of the Tracer Metric is quite stable notwithstanding some lot-to-lot variability.

Overall, this example shows Tracer Metric performs similarly to qPCR. Tracer is considerably easier to implement and allows for leveraging of historical data.

### Example 5

This example is illustrative only, and a skilled artisan will appreciate that the invention disclosed herein can be practiced in a variety of other ways.

Introduction: Donor-derived cell-free DNA (dd-cfDNA), a biomarker for kidney transplant rejection is reported as a percentage of total cfDNA. Various factors (infection, injury, age, neoplasia, and obesity) affect total cfDNA levels. We present 3 case studies with elevated background cfDNA where dd-cfDNA was assayed for rejection assessment.

Case 1: A 78 year old man with end-stage renal disease (ESRD) underwent a kidney transplant. A biopsy was performed at +6 months (m, all time points stated are relative to the date of transplant) due to an elevated creatinine level which indicated an acute T cell-mediated rejection (TCMR). At +7 m, the patient tested positive for BK viremia, which was treated. He was admitted for an elective nephrectomy of his native kidney at +14 m and tested positive for herpetic and cytomegalovirus (CMV) esophagitis for which he was treated. A cfDNA analysis at that time indicated a negative result for rejection; however, the background cfDNA level was 10,326 Arbitrary units (AU)/mL (˜21× median cfDNA).) Banff chronic active cellular rejection was confirmed from a subsequent biopsy.

Case 2: A 62 year old woman with ESRD who underwent a kidney transplant had a cfDNA assay +3 years that was reported as a negative result. However, the background was elevated at 3,466 AU/mL (˜7× median). She had a percutaneous kidney transplant biopsy that showed BK virus-associated nephropathy and TCMR.

Case 3: A 53 year old woman with ESRD had a kidney transplant from an ABO incompatible donor. A month later, she was diagnosed with dengue fever followed by acute allograft dysfunction. A biopsy at +6 m showed active antibody-mediated rejection (ABMR). On a cfDNA assay at +7 m indicated a negative result; however with an elevated background (6344 AU/mL, ˜13× median). A biopsy showed resolution of ABMR and borderline acute cellular rejection.

Discussion: In all 3 cases, active viral infections may have caused elevated total cfDNA leading to false negative results in 2 cases. A cfDNA-based rejection assay only reporting a percentage of the total cfDNA may be inaccurate, particularly in patients with viral infections. dd-cfDNA rejection assays should account for the variable background total cfDNA when reporting results.

### Example 6

This example is illustrative only, and a skilled artisan will appreciate that the invention disclosed herein can be practiced in a variety of other ways.

Introduction: Detecting elevated proportions of donor-derived cell-free DNA (dd-cfDNA) in the plasma of transplant recipients has been used as a metric to determine graft injury due to immunologic rejection. Assays that monitor rejection status report dd-cfDNA as a percentage of background cfDNA, using a cut-off of >1% to indicate rejection, and have demonstrated a sensitivity for detecting active rejection of up to 89% in clinical utility studies. However, background cfDNA levels may vary significantly in various disease states and are affected by changes in clinical and treatment-related factors. This could affect the dd-cfDNA proportion, leading to incorrect results. To clinically interpret the quantification of dd-cfDNA with respect to background cfDNA, we sought to investigate how various clinical and treatment-related factors may influence cfDNA-levels.

Objective: To investigate how various clinical and treatment-related factors may influence background cfDNA levels. To understand how to clinically interpret elevated background cfDNA levels, and to investigate how elevated levels of background cfDNA affects detection of rejection using dd-cfDNA detection.

Method: Quantification of the cfDNA amount was performed on plasma samples using next-generation sequencing and has been described before for all sample cohorts. cfDNA quantities were analyzed retrospectively for 3 different sample cohorts: kidney transplant recipients (n=1,153), pregnant women (n=20,517), early-stage cancer patients (n=1,128). Analysis of association between cfDNA concentration and patient weight, cancer type, time from surgery and treatment status was performed using absolute or indirect measures of cfDNA levels (reported as arbitrary units [AU]).

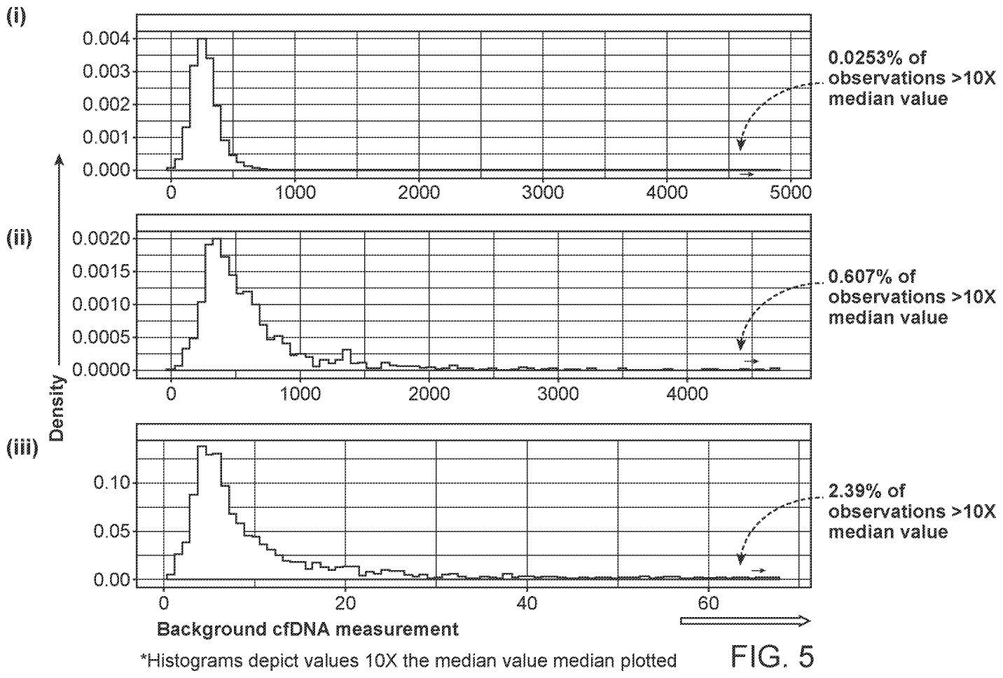

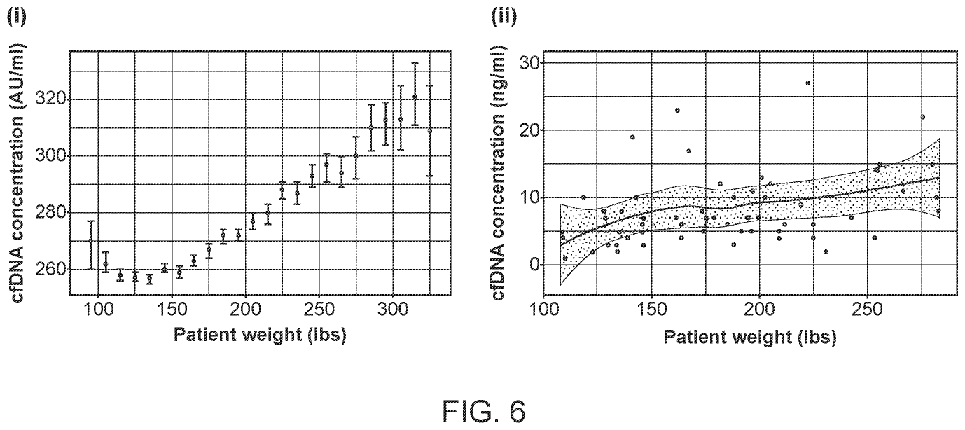

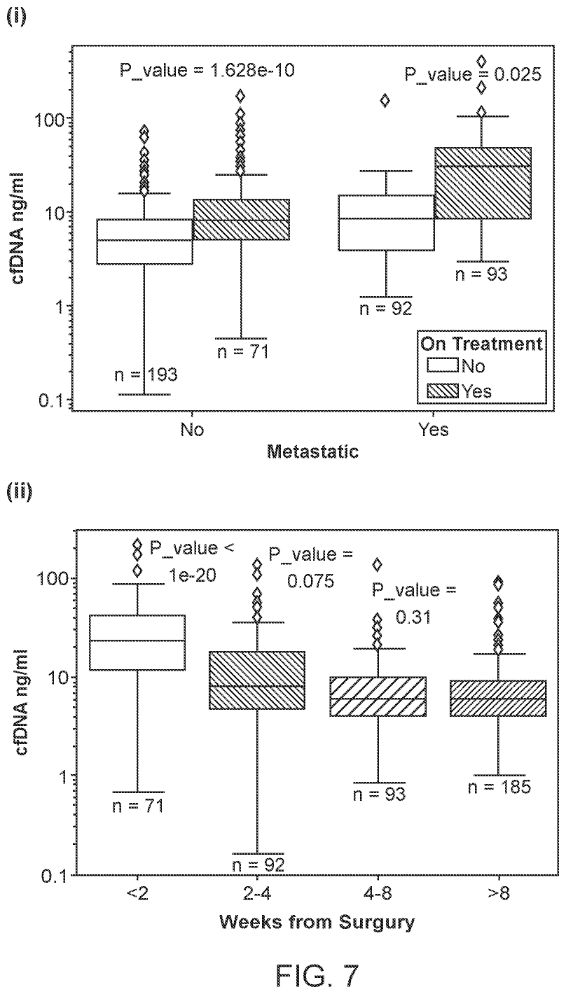

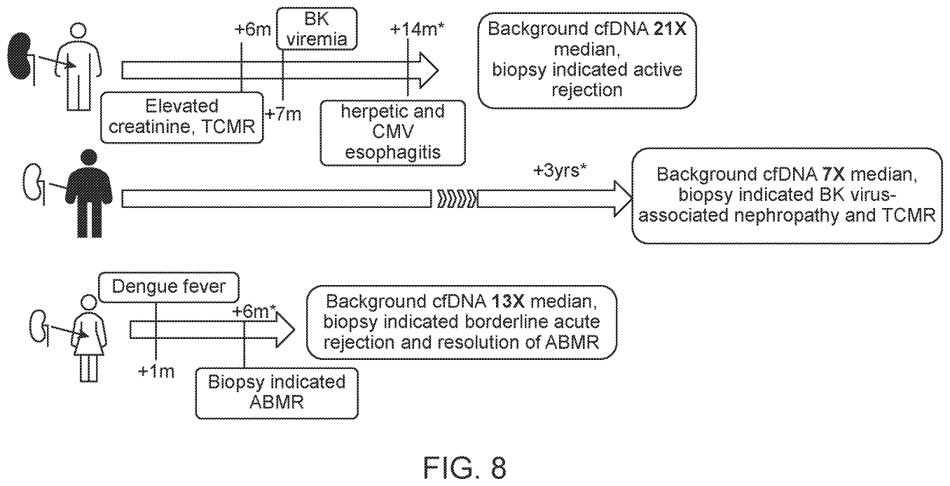

Results: Plasma cfDNA distributions in kidney transplant and early stage cancer patients (unhealthy) show a higher proportion of outliers with dramatically elevated levels of background cfDNA than pregnant women (healthy, FIG. 5). Increase in background cfDNA levels has been observed in transplant recipients undergoing active rejection. An elevated level of background cfDNA is associated with an increase in patient weight (FIG. 6). Concentration of cfDNA was significantly increased in samples collected during active treatment and metastatic cases (FIG. 7). Major trauma such as surgery leads to elevated levels of background cfDNA in plasma and is the highest within the first 2 weeks after the procedure (FIG. 7; p<0.0001). Our analysis did not reveal any statistically significant association between the level of cfDNA and patient's gender, age, and cancer type. Initial testing of dd-cfDNA with total cfDNA quantification identified 3 cases with elevations in total cfDNA varying from 7-21× median (FIG. 8).

Conclusion: Background cfDNA levels are variable and can be influenced by multiple factors, including patient weight, medications, recent surgery, body weight, viral infection, disease severity, surgical injury, and medical complications. Among kidney transplant patients, elevated background cfDNA levels may lead to false-negative results in assays using dd-cfDNA proportion as a test metric in patients with clinical or subclinical rejection. Our data indicate that patients with a viral infection may have very high background cfDNA levels which may lead to inaccuracies in dd-cfDNA assays. Dd-cfDNA-based kidney transplant rejection assays should consider both the proportion of dd-cfDNA and the background cfDNA levels when reporting results.

### Example 7

This example is illustrative only, and a skilled artisan will appreciate that the invention disclosed herein can be practiced in a variety of other ways.

Introduction: The presence of donor-derived cell-free DNA (dd-cfDNA) in blood samples from kidney transplant recipients can be utilized as a biomarker for transplant rejection. Failure of the original allograft due to rejection, infection, or recurrent disease leads to retransplants, observed in up to 10% of all kidney transplant patients. In these cases, the original transplanted kidney is generally left in-situ. A rapid, accurate, and noninvasive diagnostic test assessing dd-cfDNA using single nucleotide polymorphism (SNP) based massively multiplexed PCR (mmPCR) test (Prospera™) may be utilized to detect allograft rejection. Among retransplant patients, this test can detect both donor fractions in the plasma, when both the new and previously transplanted kidneys are releasing cfDNA.

Objective: To present the clinical performance of the SNP-based mmPCR test analysis algorithm on samples from patients with kidney retransplants in which allografts are present from two genetically distinct donors.

Materials and Methods: Plasma samples from a cohort of second transplant patients were collected and processed as described previously. The SNP-based mmPCR test algorithm is designed to detect all donor fractions in the plasma, when both the newly transplanted kidney as well as previously transplanted kidney(s) may be releasing cfDNA into the plasma. This algorithm estimates the total fraction of DNA due to all donor fractions combined.

Results: We present the clinical performance of patients with a second kidney transplant by this retransplant algorithm. In our dataset to date, no significant difference in dd-cfDNA levels compared to single allograft recipients was observed, suggesting limited cfDNA shedding from the initial kidney transplanted. Our results confirm the ability of this assay to analyze and quantify dd-cfDNA levels in kidney retransplant patients.

Conclusion: Our results indicate that performance of this SNP-based mmPCR test is preserved in repeat transplant recipients. Non-invasive assessment of dd-cfDNA in retransplant patients may be used to detect the presence of injury or rejection of the transplanted organ at an early stage, facilitating physician management around change of anti-rejection therapy.

### Example 8

This example is illustrative only, and a skilled artisan will appreciate that the invention disclosed herein can be practiced in a variety of other ways.

Introduction

Renal allograft is considered the ideal treatment for patients with end-stage kidney disease, where transplant leads to substantial improvements in patient survival and quality of life. Unfortunately, recipient mediated allograft damage and failure are common, and 20-28% of recipients are reported to experience acute kidney injury (AKI) during the transplant maintenance phase (≥3 months post-transplant), most within two years. Furthermore, −3-5% of allografts fail per year beyond the first year, with a 10-year transplant attrition rate of −55%. Chronic immunosuppression is the main treatment strategy to help prevent transplant rejection, functionally counteracting the inflammatory and immunological responses mounted by allograft recipients.

The severe acute respiratory syndrome coronavirus 2 (SARS-CoV-2), that causes COVID-19 has brought significant challenges to the treatment and management of renal transplant recipients. Chronic immunosuppression may place transplant recipients at a heightened risk of developing more severe courses of COVID-19, and virus-positive transplant recipients are known to have poorer survival outcomes compared to healthy individuals. Consequently, physicians typically lower immunosuppression in COVID-19 patients, which increases the risk of allograft rejection. Additionally, concurrent comorbidities common in kidney transplant patients, such as diabetes, obesity, and cardiac disease, are also major risk factors for severe COVID-19 symptoms and poor outcomes.

Compounding this, SARS-CoV-2 itself reportedly causes kidney damage, including acute kidney injury/failure (AKI/AKF) due to virally induced multi-organ failure, reduced renal perfusion, and cytokine storm. Kidney damage is found to increase with COVID-19 severity, and AKI/AKF are associated with poor prognosis. In severe SARS-CoV-2 infection, immunosuppressive treatments may help mitigate the cytokine storm and consequential kidney damage during the inflammatory stage of the disease. Stratification of virally infected kidney transplant patients into high- and low-risk groups for AKI/AKF could aid in physician decision making regarding patient management and treatment, including the use, dose, and timing of immunosuppressant.

Tissue biopsy is the gold standard for validating AKI/AKF and kidney transplant rejection. However, biopsy procedures are highly invasive and costly, and thus impractical for routine monitoring of kidney health. Improved biomarkers that can be used to detect AKI/AKF early and with high accuracy are greatly needed, especially in the era of COVID-19. Circulating, donor-derived cell-free DNA (dd-cfDNA) is now a proven biomarker that can detect AKI/AKF reliably, and with high sensitivity. Due to its circulation in the blood, dd-cfDNA can be measured non-invasively, and serially through a simple blood test, and is reportedly more accurate than measurement of serum creatinine. Current commercial tests generally report dd-cfDNA as a fraction of total circulating cfDNA.

Here, we present results of dd-cfDNA testing in a series of hospitalized renal allograft recipients with COVID-19, examining changes in cfDNA over time.

Methods

Patients and Samples. A retrospective analysis of dd-cfDNA test results was conducted on blood samples collected from renal allograft patients who were diagnosed with COVID-19 and had dd-cfDNA testing performed with Prospera™ (Natera, Inc.) as part of clinical care. Patients had an initial dd-cfDNA test performed shortly after infection, with a subset of patients having a follow-up test after COVID-19 clearance. Demographic, clinical and outcome data was collected for each patient and de-identified prior to analysis.

Individuals who were under 18 years of age, had more than one organ transplanted, were pregnant, or had a blood transfusion within two weeks of enrollment were excluded. The inclusion of samples in the primary analysis were based on availability of adequate plasma to run the dd-cfDNA assay, and availability of clinical follow-up.

Analysis of dd-cfDNA using mmPCR NGS assay. Blood samples were processed and analyzed at Natera, Inc.'s CLIA-Certified and College of American Pathologists (CAP) accredited laboratory (San Carlos, Calif., USA). Laboratory testing was performed using massively multiplexed-PCR (mmPCR), targeting over 13,000 single nucleotide polymorphisms. Sequencing, with an average of 10-11 million reads per sample, was performed on the Illumina HiSeq 2500 on rapid run. For all patients, both the total cfDNA level (analyzed in multiples of the median; MoM) and the donor-derived cfDNA (dd-cfDNA) fraction (analyzed as the percentage of total cfDNA) were measured.

Biopsy samples were analyzed and graded according to the standard practice at each site by their respective pathologists using Banff 2017 classification. AKI was defined as serum creatinine levels >2.0× baseline or urine output <0.5 ml/kg/h for >12 hours. Diagnosis of COVID-19 and its severity was classified based on the ordinal scale of clinical improvement published by the World Health Organization (WHO) in February, 2020.

Statistical Analyses. Differences in either total cfDNA levels or dd-cfDNA fractions were assessed between tests performed closest to the onset of COVID-19 symptoms and the follow-up time point (a proxy for baseline levels) using paired t-tests. To determine if elevated cfDNA levels are attributed to either AKI or renal replacement therapy (RRT), paired t-tests were performed across time periods and Wilcoxon rank sum tests were performed for intra-time period comparisons. Stepwise regressions were used to investigate associations of cfDNA measures (both total and dd-cfDNA) with COVID-19 severity scores (linear) and mortality (logistic regression). In addition to total cfDNA level and dd-cfDNA fraction, potential predictor variables included in these models were age, donor type and AKI. Donor type and AKI were entered as binary variables. Total cfDNA, dd-cfDNA and age were entered into models as continuous variables. Variables were entered and retained in models at P≤0.10 and P<0.15, respectively. Body Mass Index (BMI) and baseline creatinine were considered for inclusion in analyses but were inestimable in all models.

Results

Clinical Characteristics and outcomes. A total of 29 kidney transplant patients presented with COVID-19. Six of these patients were admitted to the hospital for other reasons (two for kidney transplant surgery) and contracted COVID-19 nosocomially. One patient received a kidney transplant two weeks prior to onset of COVID-19 symptoms. The median age of the cohort was 58 years (range: 21-73 years), with a median time from transplant to onset of COVID-19 of 781 days (range 6-6694). The cohort was predominantly male (62.1%), white (41.4%), with allografts received from deceased donors (79.3%).

The median time from onset of symptoms to hospital admission was 6 days, with the earliest reported onset of COVID-19 symptoms appearing 17 days before hospital admission, and the latest, 13 days after hospital admission.

AKI was diagnosed in 19 patients (65.5%). Of the 10 patients (34.4%) that required RRT, one of these individuals had no indication of AKI and three were initiated on RRT prior to COVID-19 diagnosis due to delayed graft function (DGF) following kidney transplant. Biopsies were performed on five individuals with AKI, which confirmed acute cellular rejection in two of these patients and inconclusive findings in one individual who was nonetheless treated for possible acute rejection. One patient experienced graft failure but had no signs of rejection. Twelve patients (41%) required artificial ventilation, and subsequently, seven of these patients died. The median time from onset of symptoms to death was 29 days (range: 20-53 days).

Patient Management. At the time of COVID-19 diagnosis, the most common maintenance immunosuppressants among the cohort included mycophenolate mofetil (MMF), mycophenolic acid (Myfortic), or mycophenolate sodium (MPS) for 26/29 (90%) patients; tacrolimus or envarses (tacrolimus extended release) for 23/29 (79%) patients; and prednisone for 21/29 (72%) patients. Lesser common treatments among the cohort included maintenance belatacept ( 1/29), sirolimus ( 1/29), azathioprine ( 2/29), and cyclosporine A ( 4/29). In the majority of patients, the primary change in immunosuppression was the decrease or discontinuation of MMF/MPS/Myfortic and the initiation of steroid treatment (prednisone or hydrocortisone). For treatment of COVID-19, four patients received remdesivir and/or dexamethasone, and five were administered convalescent plasma. One patient was treated with hydroxychloroquine.

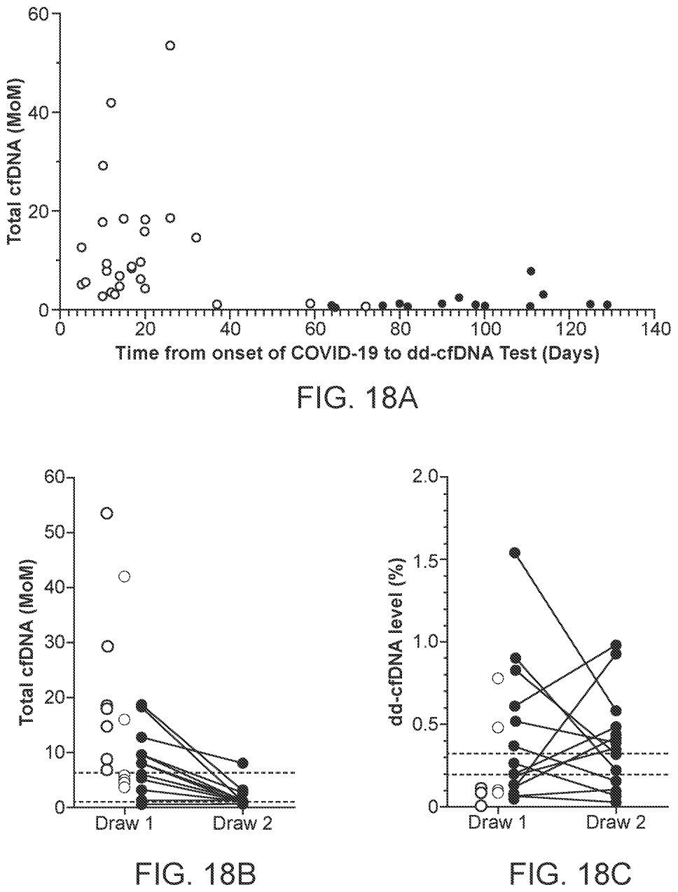

Elevated total cell free DNA levels at onset of COVID-19. Following admission to the hospital, all patients were monitored for allograft rejection using a dd-cfDNA test. For these patients, the median time from the onset of COVID-19 symptoms to the first dd-cfDNA test reading was 14 days (range: 5-72) with 25 (86%) of these tests being performed within 30 days. Fifteen of the 29 patients (51.7%) had a second follow-up dd-cfDNA test performed, after COVID-19 symptoms had subsided, with a median time of 71 days between blood draws (range: 27-112), and a median of 90 days from the onset of COVID-19 (range 64-129). Calculation of the time in days from the onset of COVID-19 to each dd-cfDNA test performed (n=44), indicated minimal overlap between the two testing periods. Comparison of these time periods and the total cfDNA values for each test revealed elevated total cfDNA levels to be present in the draws closest to the onset of COVID-19 (FIG. 18A). Further analysis revealed that 21/29 (72.4%) of initial total cfDNA readings were ≥4 MoM, and 14 (48%) were ≥8 MoM; one reading from a follow-up time point was elevated above 4 MoM.

The median total cfDNA level was substantially higher for initial tests (7.9 MoM; n=29), occurring closest to COVID-19 symptom onset compared to the follow-up tests (1.01 MoM; n=15; FIG. 18B). For the 15 patients that had two tests performed, the reading at the first time point was significantly higher (median=6.2 MoM; p=0.0009) compared to the follow-up time point (median=1.01 MoM). Additionally, total cfDNA levels decreased between the first and the second timepoints for all but one individual.

Among results from initial tests, patients who received RRT prior to first cfDNA measurement (n=7) had significantly higher total cfDNA levels (median: 17.8 MoM, range: 6.8-53.4), compared to those who did not receive RRT (n=21) (median: 5.2 MoM, range: 0.6-29.2) (P=0.01). Total cfDNA levels were similar in patients with AKI (median: 7.9 MoM, range: 0.6-53.4; n=19) and those without AKI (median: 7.4 MoM, range: 1.1-29.2; n=10) (P=0.95). We observed similar trends of decreasing cfDNA levels between the initial time point and the follow-up time point for individuals who did not receive RRT (n=13; p=0.003), who experienced AKI (n=9; p=0.01) and those who did not experience AKI (n=6; p=0.06).

The median dd-cfDNA fraction among the initial test results from the 29 patients was 0.11% (range: 0.01% to 1.54%) while the median dd-cfDNA reading for the 15 follow-up tests was 0.32% (range: 0.03% to 0.98%). Comparison of dd-cfDNA fractions for the 15 individuals with paired test results, indicated no significant difference between dd-cfDNA readings at the two timepoints (p=0.67; FIG. 18C).

Elevated total cfDNA levels obscured indication of rejection by dd-cfDNA testing. Biopsy showed acute cellular rejection in two individuals in our cohort. Tests from the initial time points indicated dd-cfDNA fractions of 0.2% and 0.48, accompanied by total cfDNA levels of 7.9 MoM and 41.8 MoM, respectively. For the first individual, biopsy-confirmed rejection occurred ten days after their initial dd-cfDNA test. This patient experienced decreases in total cfDNA levels to 0.60 MoM accompanied by a dd-cfDNA fraction of 0.48% at the follow-up time point, after treatment of the rejection. For the second individual, biopsy-confirmed rejection occurred 72 days after dd-cfDNA testing. Follow-up dd-cfDNA testing was not performed for this individual.

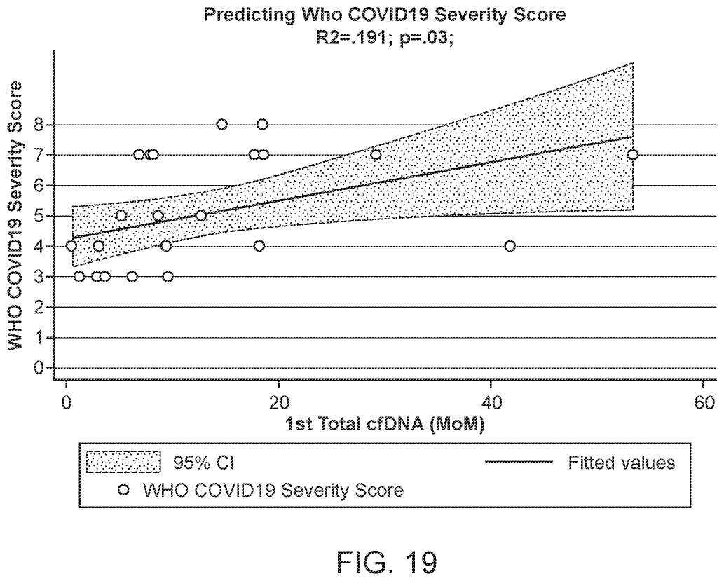

Total cfDNA levels are associated with COVID-19 severity. Clinical COVID-19 severity scores in this cohort ranged from 3 (indicating hospitalization with no oxygen therapy) to 8 (indicating mortality) on a scale from 1 to 8, with a median score of 5. Stepwise regression identified a significant positive association between total cfDNA levels and the COVID-19 severity score (P=0.03; R²=0.19; FIG. 19). No other covariates achieved the P≤0.10 level of significance required for inclusion in the model.

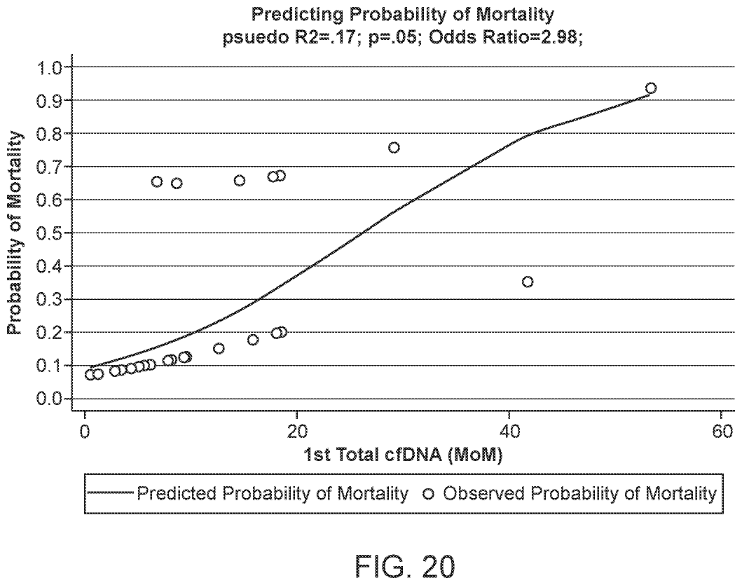

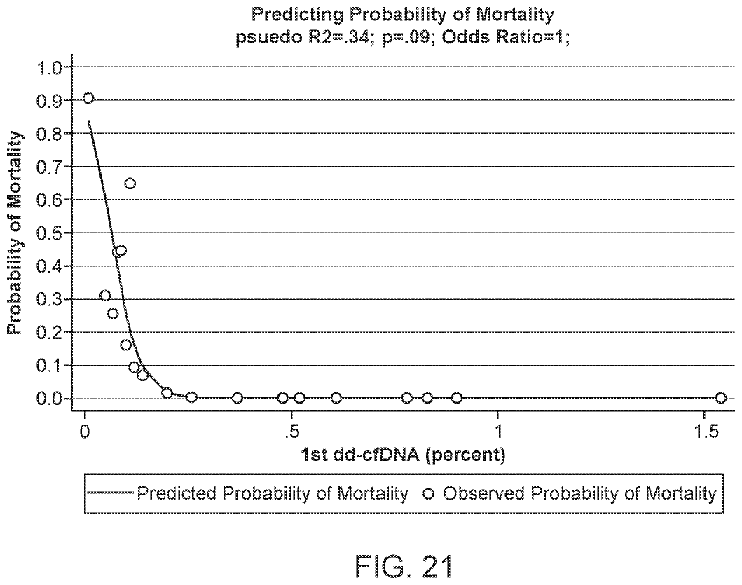

Decreased dd-cfDNA levels are associated with probability of death from COVID-19. Stepwise regression analysis selected total cfDNA and dd-cfDNA as the only predictors of mortality. Neither of these variables were statistically significant at the P<0.05 level (P=0.08 for both, total cfDNA and dd-cfDNA). The probability of death increased with increasing total cfDNA levels (FIG. 20). In contrast, the probability of death increased as dd-cfDNA fraction decreased, but only for dd-cfDNA values less than 0.25%. Above 0.25%, probability of death was estimated to be 0 (FIG. 21).

Discussion

SARS-CoV-2 infection is especially dangerous to patients with a renal allograft. First, it has been shown to strongly correlate with AKI, and second, immunosuppression is typically tapered during infection to enable immune responses against the virus, which increases the risk of rejection. cfDNA is an emerging non-invasive marker for monitoring allograft injury and risk of rejection. Here, we analyzed total cfDNA levels and dd-cfDNA fractions in 29 hospitalized renal allograft patients with COVID-19. We followed up with a subset of patients, tracking changes in dd-cfDNA and total cfDNA levels approximately two months after the initial test.

Total cfDNA levels were highly elevated in patients at the time of their first test, close to the onset of COVID-19. In this cohort, 75% and 48% of total cfDNA readings from initial tests were elevated above 4 and 8 MoM, compared to 4.8% and 1.2%, respectively, in a cohort of unselected kidney transplant recipients who received dd-cfDNA testing during routine care. This is consistent with literature showing a correlation between total cfDNA and viral infection. We also observed a significant decrease in total cfDNA levels, with only one reading (6.7%)≥4 MoM at the follow-up time point, after patients are expected to have recovered from the COVID-19. Additionally, 14 of the 15 patients for whom two tests were performed experienced decreases in their total cfDNA levels between time points. This trend is in line with a recent case study wherein a single kidney transplant recipient with COVID-19 had total cfDNA levels elevated to 57 MoM during infection, with levels declining to 2.9 MoM over the course of one and a half months, during clearance of the infection.

In this cohort, the majority of the samples with elevated total cfDNA levels were drawn within 32 days of the onset of COVID-19 symptoms. Reports indicate that the median duration of positivity for SARS-CoV-2 is approximately 20 days, and can last as long as 53 days, in a general population. The infection has been observed to last significantly longer in immunocompromised and organ transplant patients, as well as critically ill patients, with approximately 60% of patients clearing the virus within 30 days. As all tests at the follow-up time point occurred >60 days after COVID-19 onset. Thus, these data support the hypothesis that the elevated cfDNA levels seen within 32 days of symptom onset were caused by active SARS-CoV-2 infection.

Our analysis also demonstrated a significant correlation between total cfDNA levels and COVID-19 severity, corroborating another study that similarly identified an association between cfDNA concentrations and WHO clinical progression scores in hospitalized patients. We also found that initial total cfDNA levels, measured during the peak of symptom severity, were higher in all subsets of individuals queried, including those requiring or not requiring RRT, and patients with and without AKI. Although studies have implicated RRT such as hemodialysis in elevations in cfDNA, our findings suggest that RRT cannot fully account for the changes observed. Additionally, in our analysis, differences in cfDNA levels between individuals with and without AKI were not significant, indicating that this variable also did not account for the elevated total cfDNA levels. This provides additional evidence that the SARS-CoV-2 infection contributed substantially to the initial elevated cfDNA levels we observed.

In contrast to the total cfDNA levels, we did not observe an increase in dd-cfDNA levels at the first time point, when patients were experiencing COVID-19 symptoms. This is not surprising, as elevations in total cfDNA levels would be expected to depress the proportion of dd-cfDNA. Indeed, only one patient (3.4%) had dd-cfDNA levels above the 1% threshold for indication of allograft injury/rejection, as compared to clinical cohorts which typically have detection rates of −10% in clinically stable patients, and −25% in patients with a clinical suspicion of rejection.

Two individuals in our cohort were found to have active rejection by biopsy; both of these individuals had elevated total cfDNA and dd-cfDNA levels <1% at the first time point, suggesting that in these cases, the elevated total cfDNA may have confounded the dd-cfDNA results. For both patients, the tests resulting in elevated cfDNA levels occurred 11 and 12 days following onset of COVID-19, and thus were likely actively infected at the times of these tests. Other studies have suggested that quantification of the absolute dd-cfDNA concentrations was a more valuable marker in assessing allograft rejection, as representing dd-cfDNA as a fraction of total levels can mask subtle but important changes in the amount of dd-cfDNA released from allografts. Accounting for absolute concentration of dd-cfDNA could, thus, provide better detection of allograft rejection, particularly under conditions when total cfDNA levels may be affected, including viral infections such as COVID-19.

We conclude that an elevation in total cfDNA is associated with COVID-19 in hospitalized kidney transplant patients, and that total cfDNA levels are correlated with COVID-19 severity. Additionally, dd-cfDNA testing remains a useful non-invasive tool for monitoring allograft rejection in individuals critically ill with COVID-19, and for informing the need for more invasive procedures such as biopsy. It is important to consider total cfDNA levels, along with the dd-cfDNA fraction, in management of individuals who may have viral infections.

### Example 9

This example is illustrative only, and a skilled artisan will appreciate that the invention disclosed herein can be practiced in a variety of other ways.

Elevated total cfDNA occurring during viral infection such as COVID-19 (see Examples 5 and 8) may lead to false negatives in a dd-cfDNA assay that relies on estimated percentage of dd-cfDNA as the sole cutoff threshold to indicate transplant rejection. To improve sensitivity and accuracy of the dd-cfDNA assay and reduce false negatives in the presence of high total cfDNA in plasma samples, an additional cutoff threshold ADDD was added, which is proportional to the absolute donor-derived DNA concentration. The additional cutoff threshold can be calculated as ADDD=estimated dd-cfDNA %×(total sample sequence reads/Tracer sequence reads/plasma volume).

Both dd-cfDNA % and ADDD were applied to analyze plasma samples from kidney transplant recipients suffering from active viral infection. Compared to relying on estimated dd-cfDNA % alone (e.g., call rejection if dd-cfDNA %>1%), incorporating the additional cutoff threshold described above (e.g., call rejection if estimated dd-cfDNA %>1% or ADDD>6.9 ml) significantly reduced false negatives and improved sensitivity and accuracy of the dd-cfDNA assay.

### Example 10

This example is illustrative only, and a skilled artisan will appreciate that the invention disclosed herein can be practiced in a variety of other ways. This example demonstrates detection of rejection in kidney transplant patients using an algorithm that combines donor fraction and absolute dd-cfDNA.

Donor-derived cell-free DNA (dd-cfDNA) in the plasma of renal allograft patients is a clinically validated biomarker for allograft injury and rejection. Several dd-cfDNA assays have shown that >1% dd-cfDNA is associated with a high risk for active rejection (AR). Additional studies have shown the advantage of measuring absolute dd-cfDNA concentration to avoid the variability that dd-cfDNA fraction encounters due to the host-derived cfDNA component. Presented here are results from a new algorithm that combines both dd-cfDNA donor fraction and absolute amount of dd-cfDNA (ADD-cfDNA) in the plasma, and the results were compared with previous algorithm.

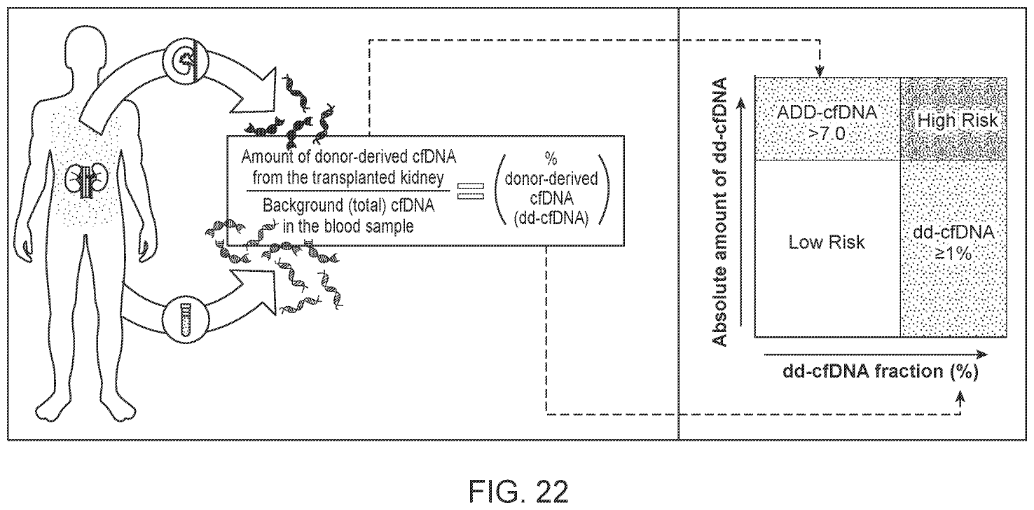

40 plasma samples were collected from kidney transplant recipients as a part of routine clinical care. Matched biopsy samples were obtained, where available, and were defined as: a) AR, with TCMR and/or ABMR rejection, and b) clinically stable. Performance of the two-threshold algorithm was estimated using the previously validation dd-cfDNA fraction cutoff (≥1%) and a second cut-off based on the ADD-cfDNA (>7.0) (FIG. 22). Samples that exceeded either the 1% dd-cfDNA fraction or the new ADD-cfDNA cut-off were considered high risk for rejection. The performance of the updated algorithm was compared to the previous algorithm that used the 1% dd-cfDNA fraction threshold alone.

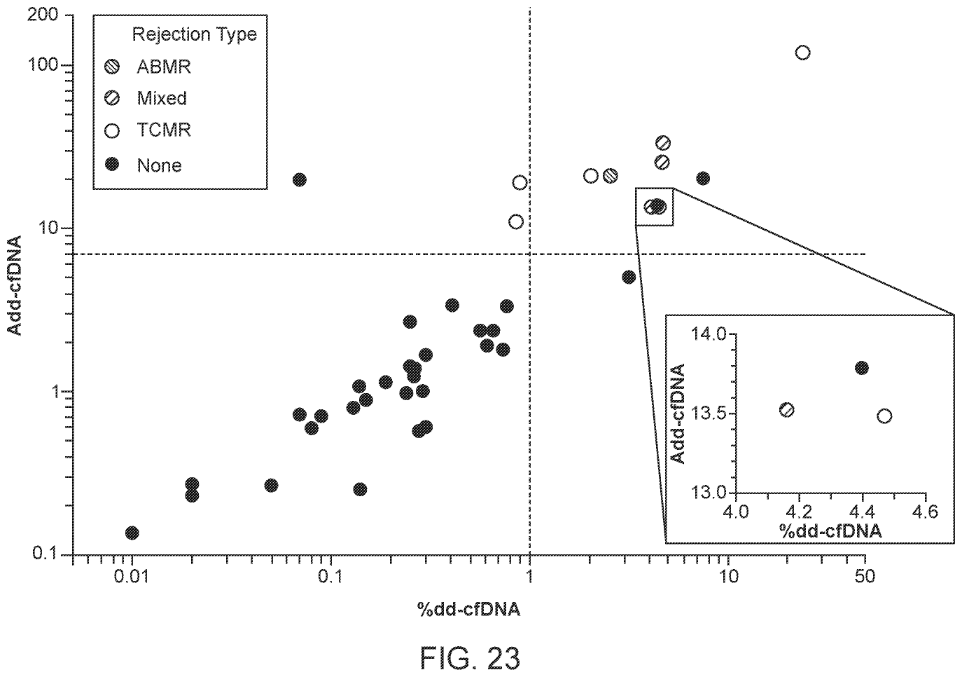

Six patients had TCMR (2×IA, 2×IB, 1×IIB), one had ABMR and two had a mixed rejection. As shown in FIG. 23, the updated algorithm demonstrated improved performance, with an observed sensitivity of 9/9 (100%), as compared to the previous algorithm with a 1% dd-cfDNA threshold, 7/9 (77.8%), without compromising the specificity (90.3%; 28/31). In conclusion, host-derived cfDNA can be influenced by a number of physiological and pathological factors, which can affect the reported dd-cfDNA fraction and potentially decrease test accuracy. An algorithm that incorporates absolute amounts of dd-cfDNA with dd-cfDNA fraction is clinically meaningful as it increases sensitivity in detecting rejection in renal allograft patients without affecting the specificity.

### Example 11

This example is illustrative only, and a skilled artisan will appreciate that the invention disclosed herein can be practiced in a variety of other ways. This example demonstrates detection of rejection in kidney transplant patients using an algorithm that combines donor fraction and absolute dd-cfDNA.

Donor-derived cell-free DNA (dd-cfDNA) in the plasma of renal allograft patients is a clinically validated biomarker for allograft injury and rejection. Several studies have shown that ≥1% dd-cfDNA is associated with a high risk for active rejection (AR). Other studies reported the advantage of measuring absolute dd-cfDNA concentration to avoid changes in dd-cfDNA fraction due to the variability of the host-derived cfDNA component. Presented here are results from a new two-threshold algorithm that combines both dd-cfDNA donor fraction and absolute concentration of dd-cfDNA in the plasma and compare results with previous algorithm.

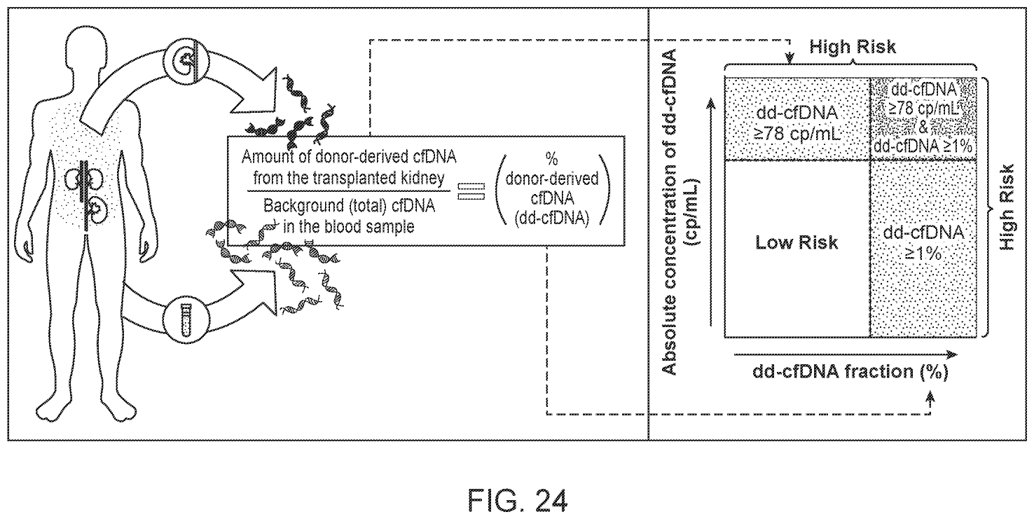

41 plasma samples were collected from kidney transplant recipients as a part of routine clinical care. Matched biopsy samples were obtained, where available, and were defined as: a) AR, with TCMR and/or ABMR rejection, and b) clinically stable. Performance of the two-threshold algorithm was estimated using the previous validated dd-cfDNA fraction cutoff (≥1%) and a second cut-off based on the absolute concentration of dd-cfDNA (≥78 copies/mL) (FIG. 24). Samples that exceeded either the 1% dd-cfDNA or the new 78 cp/mL dd-cfDNA cut-offs were considered high risk for rejection. The performance of the updated algorithm was compared to the previous algorithm that used the 1% dd-cfDNA fraction threshold alone.

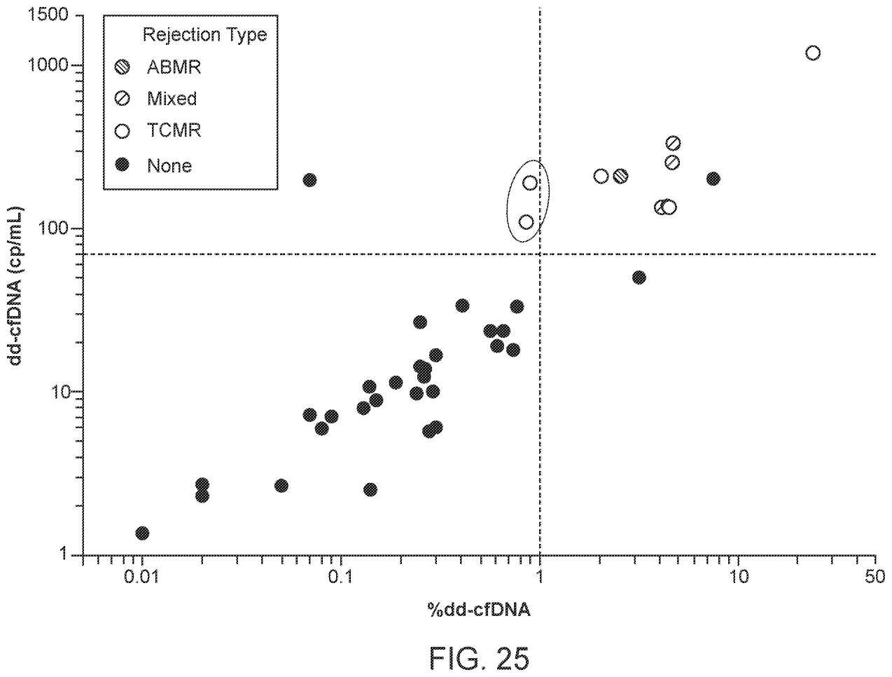

Five patients had TCMR (2×IA, 2×IB, 1×IIA), one had ABMR and three had a mixed rejection. Sensitivity of the two-threshold algorithm was 9/9 (100%), compared to 7/9 (77.8%) with previous algorithm (1% dd-cfDNA threshold). Specificity of the updated and previous algorithms was 28/32 (87.5%) and 29/32 (90.6%), respectively (FIG. 25). In conclusion, host-derived cfDNA can be influenced by a number of physiological and pathological factors, including COVID-19, which can affect the reported dd-cfDNA fraction, potentially decreasing test accuracy. An algorithm incorporating absolute concentration of dd-cfDNA with dd-cfDNA fraction is clinically meaningful as it increases sensitivity in detecting rejection in renal allograft patients.

1. A method or laboratory technique, comprising:
a) isolating cell-free DNA from a biological sample, wherein a first Tracer DNA composition is added before or after isolation of the cell-free DNA;
b) performing targeted amplification of the isolated cell-free DNA at 100 or more different target loci in a single reaction volume using 100 or more different primer pairs;
c) sequencing the amplification products by high-throughput sequencing to generate one or more sequencing reads; and
d) quantifying the amount of total cell-free DNA using sequencing reads derived from the first Tracer DNA composition.

2. A method or laboratory technique, comprising:
a) isolating cell-free DNA from a biological sample of the transplant recipient, wherein the isolated cell-free DNA comprises donor-derived cell-free DNA and recipient-derived cell-free DNA, wherein a first Tracer DNA composition is added before or after isolation of the cell-free DNA;
b) performing targeted amplification of the isolated cell-free DNA at 100 or more different target loci in a single reaction volume using 100 or more different primer pairs;
c) sequencing the amplification products by high-throughput sequencing to generate one or more sequencing reads; and
d) quantifying the amount of donor-derived cell-free DNA and the amount of total cell-free DNA, wherein the amount of total cell-free DNA is quantified using sequencing reads derived from the first Tracer DNA composition.

3. The method of claim 2, wherein the method further comprises determining the occurrence or likely occurrence of transplant rejection using the amount of donor-derived cell-free DNA.

4. The method of claim 3, wherein the amount of donor-derived cell-free DNA is compared to a cutoff threshold value to determine the occurrence or likely occurrence of transplant rejection, wherein the cutoff threshold value is determined according to the amount of total cell-free DNA.

5. The method of claim 4, wherein the cutoff threshold value is a function of the number of reads of the donor-derived cell-free DNA.

6. The method of any of the preceding claims, wherein the method further comprises flagging the sample if the amount of total cell-free DNA falls outside a pre-determined range.

7. The method of any of claims 1-6, wherein the method comprising adding the first Tracer DNA composition to a whole blood sample before plasma extraction.

8. The method of any of claims 1-6, wherein the method comprising adding the first Tracer DNA composition to a plasma sample after plasma extraction and before isolation of the cell-free DNA.

9. The method of any of claims 1-6, wherein the method comprising adding the first Tracer DNA composition to a composition comprising the isolated cell-free DNA.

10. The method of any of claims 1-6, wherein the method comprises ligating adaptors to the isolated cell-free DNA to obtain a composition comprising adaptor-ligated DNA, and adding the first Tracer DNA composition to the composition comprising adaptor-ligated DNA.

11. The method of any of claims 1-10, wherein the method further comprises adding a second Tracer DNA composition before the targeted amplification.

12. The method of any of claims 1-10, wherein the method further comprises adding a second Tracer DNA composition after the targeted amplification.

13. The method of any of the preceding claims, wherein the first and/or second Tracer DNA composition comprises a plurality of DNA molecules having different sequences.

14. The method of any of the preceding claims, wherein the first and/or second Tracer DNA composition comprises a plurality of DNA molecules having different concentrations.

15. The method of any of the preceding claims, wherein the first and/or second Tracer DNA composition comprises a plurality of DNA molecules having different lengths.

16. The method of any of the preceding claims, wherein the first and/or second Tracer DNA composition comprises a plurality of DNA molecules having sequences of non-human origin.

17. The method of any of the preceding claims, wherein the first and/or second Tracer DNA composition comprises a plurality of DNA molecules of non-human origin.

18. The method of any of the preceding claims, wherein the first and/or second Tracer DNA composition comprises a plurality of DNA molecules having artificial sequences.

19. The method of claim 15, wherein the plurality of DNA molecules having different lengths are used to determine size distribution of the cell-free DNA in the sample.

20. The method of any of the preceding claims, wherein the first and/or second Tracer DNA composition each comprises a target sequence, wherein the target sequence comprises a barcode positioned between a pair of primer binding sites capable of binding to one of the primer pairs.

21. The method of claim 20, wherein the barcode comprises reverse complement of a corresponding endogenous genomic DNA sequence capable of being amplified by the same primer pair.

22. The method of any of claims 20-21, wherein the ratio between the number of reads of the Tracer DNA and the number of reads of sample DNA is used to quantify the amount of total cell-free DNA.

23. The method of any of claims 20-21, wherein the ratio between the number of reads of the barcode and the number of reads of the corresponding endogenous genomic DNA sequence is used to quantify the amount of total cell-free DNA.

24. The method of any of claims 20-23, wherein the target sequence is flanked on one or both sides by endogenous genomic DNA sequences.

25. The method of any of the preceding claims, wherein the first and/or second Tracer DNA composition comprises synthetic double-stranded DNA molecules.

26. The method of any of the preceding claims, wherein the first and/or second Tracer DNA composition comprises DNA molecules having a length of 250-500 bp.

27. The method of any of the preceding claims, wherein the first and/or second Tracer DNA composition comprises DNA molecules having a length of 100-250 bp.

28. The method of any of the preceding claims, wherein the first and/or second Tracer DNA composition comprises DNA molecules having a length of 125-200 bp.

29. The method of any of the preceding claims, wherein the first and/or second Tracer DNA composition comprises DNA molecules having a length of about 160 bp.

30. The method of any of the preceding claims, wherein the targeted amplification comprises amplifying at least 100 SNP loci in a single reaction volume.

31. The method of any of the preceding claims, wherein the targeted amplification comprises amplifying at least 1,000 SNP loci in a single reaction volume.

32. The method of any of the preceding claims, wherein the targeted amplification comprises amplifying at least 10,000 SNP loci in a single reaction volume.

33. The method of any of the preceding claims, wherein each primer pair is designed to amplify a target sequence of about 35 to 200 bp.

34. The method of any of the preceding claims, wherein each primer pair is designed to amplify a target sequence of about 50-100 bp.

35. The method of any of the preceding claims, wherein each primer pair is designed to amplify a target sequence of about 60-75 bp.

36. The method of any of the preceding claims, wherein the transplant recipient is a human subject.

37. The method of any of the preceding claims, wherein the transplant is organ transplant, tissue transplant, or cell transplant.

38. The method of any of the preceding claims, wherein the transplant is kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, or blood transfusion.

39. The method of any of the preceding claims, further comprising determining the transplant rejection as antibody mediated transplant rejection, T-cell mediated transplant rejection, graft injury, viral infection, bacterial infection, or borderline rejection.

40. The method of any of the preceding claims, further comprising quantifying the likelihood of one or more cancers.

41. The method of claim 40, further comprising quantifying the susceptibility of any likely cancers to particular treatments.

42. The method of any of the preceding claims, further comprising determining the likelihood of a viral infection.

43. The method of any of the preceding claims, further comprising determining the likelihood of a bacterial infection.

44. The method of any of the preceding claims, further comprising determining the likelihood of inflammation stemming from an injury.

45. The method of any of the preceding claims, wherein the method is performed without prior knowledge of donor genotypes.

46. The method of any of the preceding claims, wherein the biological sample is a blood, plasma, serum, solid tissue, or urine sample.

47. The method of any of the preceding claims, further comprising longitudinally collecting a plurality of biological samples from the transplant recipient, and repeating steps (a) to (d) for each sample collected.

48. The method of any of the preceding claims, wherein the isolated cell-free DNA comprises donor-derived cell-free DNA derived from a first donor, donor-derived cell-free DNA derived from a second donor, and recipient-derived cell-free DNA.

49. A method of utilizing a Tracer DNA to quantitate cell-free DNA in a biological sample, comprising:
a) isolating cell-free DNA from the biological sample, wherein a first Tracer DNA composition is added before or after isolation of the cell-free DNA;
b) performing targeted amplification of said cell-free DNA at 100 or more different target loci in a single reaction volume using 100 or more different primer pairs;
c) sequencing the amplification products by high-throughput sequencing to generate one or more sequencing reads; and
d) quantifying the amount of total cell-free DNA using said sequencing reads derived from the first Tracer DNA composition.

50. A method or laboratory technique, comprising:
a) isolating cell-free DNA from a biological sample of a retransplant recipient, wherein the isolated cell-free DNA comprises donor-derived cell-free DNA derived from a first donor, donor-derived cell-free DNA derived from a second donor, and recipient-derived cell-free DNA;
b) performing targeted amplification of said cell-free DNA at 100 or more different target loci in a single reaction volume using 100 or more different primer pairs;
c) sequencing the amplification products by high-throughput sequencing to generate one or more sequencing reads; and
d) quantifying the amount of total donor-derived cell-free DNA, the amount of donor-derived cell-free DNA from the first donor, and/or the amount of donor-derived cell-free DNA from the second donor.

51. The method of claim 3, wherein the transplant recipient has an elevated amount of background cell-free DNA.

52. The method of claim 51, wherein the elevated amount of total cell-free DNA is caused by active viral infection.

53. The method of claim 52, wherein viral infection is COVID-19.

54. The method of any of claims 1-53, wherein the method comprises determining the occurrence or likely occurrence of transplant rejection using a first and a second cutoff thresholds.

55. The method of claim 54, wherein the first cutoff threshold is an estimated percentage of donor-derived cell-free DNA out of total cell-free DNA.

56. The method of claim 54, wherein the second cutoff threshold is proportional to the absolute donor-derived cell-free DNA concentration.

57. The method of claim 54, wherein the second cutoff threshold is calculated by multiplying the first cutoff threshold with a quant, wherein the quant is calculated by dividing the number of reads of total cell-free DNA by the number of reads of Tracer DNA per plasma volume.

58. The method of claim 54, wherein the first cutoff threshold is an estimated percentage of donor-derived cell-free DNA out of total cell-free DNA, wherein the second cutoff threshold is calculated by multiplying the first cutoff threshold with a quant, wherein the quant is calculated by dividing the number of reads of total cell-free DNA by the number of reads of Tracer DNA per plasma volume.

59. The method of claim 54, wherein the first cutoff threshold is an estimated percentage of donor-derived cell-free DNA out of total cell-free DNA, wherein the second cutoff threshold is a concentration of donor-derived cell-free DNA.

60. The method of any of claims 54-59, wherein the method comprises calling the occurrence or likely occurrence of transplant rejection if the amount of donor-derived cell-free DNA exceeds the first cutoff threshold or the second cutoff threshold.

61. The method of any of claims 1-53, wherein the method comprises determining the occurrence or likely occurrence of transplant rejection using a cutoff thresholds, wherein the cutoff threshold is a function of the amount of donor-derived cell-free DNA and the amount of total cell-free DNA; or wherein the cutoff threshold is a function of the number of reads of donor-derived cell-free DNA and the number of reads of total cell-free DNA.

62. The method of any of claims 1-53, wherein an estimate percentage of donor-derived cell-free DNA is used in combination with a measurement of the total cell-free DNA concentration to determine the likelihood of organ failure.

63. The method of any of claims 1-53, wherein an absolute donor-derived cell-free DNA concentration or a function thereof is used in combination with a measurement of the total cell-free DNA concentration to determine the likelihood of organ failure.

64. A method for amplifying and sequencing DNA, comprising:
(a) extracting DNA from a blood sample of a transplant recipient, wherein the DNA comprises donor-derived cell-free DNA and recipient-derived cell-free DNA;
(b) performing targeted amplification at 500-50,000 target loci in a single reaction volume using 500-50,000 primer pairs to obtain amplicons;
(c) sequencing the amplicons by high-throughput sequencing; and
(d) quantifying an absolute amount of donor-derived cell-free DNA and a percentage of donor-derived cell-free DNA out of total cell-free DNA, wherein (i) the percentage of donor-derived cell-free DNA or a function thereof above a first threshold and/or (ii) the absolute amount of donor-derived cell-free DNA or a function thereof above a second threshold is indicative of transplant rejection.

65. A method for amplifying and sequencing DNA, comprising:
(a) extracting DNA from a blood sample of a kidney transplant recipient, wherein the DNA comprises donor-derived cell-free DNA and recipient-derived cell-free DNA;
(b) performing targeted amplification at 500-50,000 target loci in a single reaction volume using 500-50,000 primer pairs to obtain amplicons;
(c) sequencing the amplicons by high-throughput sequencing; and
(d) quantifying an absolute amount of donor-derived cell-free DNA and a percentage of donor-derived cell-free DNA out of total cell-free DNA, wherein (i) the percentage of donor-derived cell-free DNA above 1% and/or (ii) the concentration of donor-derived cell-free DNA above 78 copies/ml is indicative of kidney transplant rejection.

66. A method for amplifying and sequencing DNA, comprising:
(a) extracting DNA from a blood sample of a kidney transplant recipient, wherein the DNA comprises donor-derived cell-free DNA and recipient-derived cell-free DNA;
(b) performing targeted amplification at 500-50,000 target loci in a single reaction volume using 500-50,000 primer pairs to obtain amplicons;
(c) sequencing the amplicons by high-throughput sequencing; and
(d) quantifying an absolute amount of donor-derived cell-free DNA and a percentage of donor-derived cell-free DNA out of total cell-free DNA, wherein (i) the percentage of donor-derived cell-free DNA above 1% and/or (ii) a function of the absolute amount of donor-derived cell-free DNA above 7.0 is indicative of kidney transplant rejection, said function of the absolute amount of donor-derived cell-free DNA is calculated by multiplying the percentage of donor-derived cell-free DNA with the number of reads of total cell-free DNA divided by the number of reads of Tracer DNA per plasma volume.
